(19) United States
(12) Patent Application Publication
(10) Pub. No.: US 2023/0293771 A1
(43) Pub. Date: Sep. 21, 2023

(54) CHONDROCYTE CELL SHEETS AND METHODS FOR THEIR PRODUCTION AND USE

(71) Applicants: UNIVERSITY OF UTAH RESEARCH FOUNDATION, Sailt Lake city, CT (US); ORGANIZATION OF CELL SHEET TISSUE ENGINEERING REGENERATIVE MEDICINE INITIATIVES, Tokyo (JP)

(72) Inventors: Mimi Egami, Tokyo (JP); David Grainger, Sailt Lake city, UT (US); Kyungsook Kim, Murray, UT (US); Makoto Kondo, Sailt Lake city, UT (US); Travis Maak, Park City, UT (US); Teruo Okano, Sailt Lake city, UT (US); Hideaki Sakai, Kawasaki-shi, Kanagawa (JP)

(73) Assignees: UNIVERSITY OF UTAH RESEARCH FOUNDATION, Sailt Lake city, UT (US); ORGANIZATION OF CELL SHEET TISSUE ENGINEERING REGENERATIVE MEDICINE INITIATIVES, Tokyo (JP)

(21) Appl. No.: 18/016,491

(22) PCT Filed: Jul. 16, 2021
(86) PCT No.: PCT/US2021/041942

§ 371 Date: Jan. 17, 2023

Related U.S. Application Data
(60) Provisional application No. 63/052,496, filed on Jul. 16, 2020.

(51) Int. Cl. A61L 27/38; C12N 5/077; C12N 11/02; A61L 27/36

(52) U.S. Cl. CPC A61L 27/3852; C12N 5/0655; C12N 11/02; A61L 27/3817; A61L 27/3691; C12N 2509/00; C12N 2533/90; C12N 2513/00; C12N 2533/54; A61L 2430/06

(57) ABSTRACT

The disclosure provides a chondrocyte cell sheet comprising one or more layers of confluent cells comprising chondrocytes and chondroprogenitor cells. Methods of generating cartilage tissue in a subject are also provided. The disclosure also provides a method for producing chondrocyte cell sheets comprising culturing chondrocytes and chondroprogenitor cells in culture solution on a temperature-responsive polymer which has been coated onto a substrate surface of a cell culture support, wherein the temperature-responsive polymer has a lower critical solution temperature in water of 0-80° C.; adjusting the temperature of the culture solution to below the lower critical solution temperature, whereby the substrate surface is made hydrophilic and adhesion of the cell sheet to the surface is weakened; and detaching the cell sheet from the culture support.

### RELATED APPLICATIONS

This application claims priority to U.S. Provisional Patent Application No. 63/052,496 filed on Jul. 16, 2020, the contents of which are incorporated herein in their entirety.

### BACKGROUND OF THE INVENTION

Due to improved access to qualified healthcare services, average life span has been extended significantly in the developed world, which has resulted in a 76.0 year average life-span for males and 81.0 for females in the US, and 81.1 for males and 87.1 for females in Japan. (2016 data of average life span, published by 2018 WHO report). To optimize total quality of life for people living in the aging society, and reduce the burden of increased cost of medical care and medicaid services, so-called “locomotive syndrome” disease is becoming a serious target of global health policy, which disease reduces mobility of patient's daily life and requires assistant care, in both traumatic and chronic condition, due to impairment of locomotive organs such as knee cartilage. The current number of patients for knee osteoarthritis and degenerative lumbar spondylopathy are 25 million and 38 million in Japan, and approximately 32 million osteoarthritis patients in the US. The total number of patients in the field of “locomotive syndrome” has already reached 47 million in Japan. Medical outcomes of standard treatments, such as steroid injection, microfracture, or artificial joint replacement, are quite limited in longer term prognostic to regenerate knee cartilage, i.e. to cure locomotive function and regain quality of life for osteoarthritis and other knee cartilage locomotive diseases patients especially in the case of aging patients.

In order to regenerate knee cartilage, which is not self-healing tissue because of its avascularity, preclinical and clinical studies have evaluated various methods such as injection of mesenchymal stem cells, transplantation of adult chondrocyte cells or cell units cultured and manipulated from autologous and allogeneic cartilage tissue, or transplantation of cadaver knee cartilage tissue with fibrin gel. “Carticell”, cultured autologous chondrocyte cell product for transplantation by Genzyme Co., was a first approved cell therapy product by FDA to transplant patient's own chondrocyte cells to a defect site. However injected cells are often leaked out from the site or unevenly distributed by gravity, and adhered cells often lose substrate production ability before the hyaline structure is generated. These approaches did not result in sufficient regeneration of the patient's own hyaline cartilage tissue at the targeted deficient site, and hyaline cartilage is the main component of cartilage to bear human body weight and maintain locomotive function to maintain the quality of life.

Teruo Okano et al. in Japan (at Tokyo Women's Medical University) generated the concept of “cell sheet engineering”, which isolates target cells from patient's own tissue, cultures cells as a sheet-formed cell unit by using thermo-responsive culture surfaces, and then harvests the cell sheet, and transplants the sheet to a target site without damaging the adhesive intact membrane protein, cell-cell junction and extracellular matrix (ECM). This group has succeeded in human clinical application of cultured cell sheets to treat corneal epithelia, heart, esophageal epithelia, periodontal ligament, as well as knee cartilage.

As a part of the cell sheet engineering approach discussed above, Prof Masato Sato et al. in Japan (Tokai University, US2008/0226692) applied autologous “cell sheet engineering” to regenerate knee cartilage by collecting a biopsy of an adult patient's own knee cartilage tissue from the non-loaded part, isolate chondrocyte cells, and cultured cell sheets to transplant onto the defective part of knee cartilage. The cell sheets were positive for immunostaining using an antibody against type II collagen and negative against type I collagen. Cells obtained by biopsy from the patients showed good biocompatibility, however the adult cells could not be cultured right away and over a long time period. As a result, the volume of cells was quite limited, and patients could not be treated immediately because of the culture period for the cells. Cell sheets generated from allogeneic cell sources with sufficient biocompatibility is highly expected.

Masato Sato et al. (US2018/0243476) investigated the potential of polydactyly-derived infant cartilage tissue as an allogeneic chondrocyte cell source, since excised fingers/toes are normally disposed of immediately after surgery. The cell sheets were negative for immunostaining using an antibody against type II collagen and positive against type I collagen. They performed a human study to isolate chondrocyte cells from excised fingers and toes and generated allogeneic cell sheets to regenerate the deficient surface of a patient's knee cartilage. However, their infant polydactyly-derived chondrocyte cells are highly undifferentiated and immature, therefore their generated cell sheets were not produced easily or with constant quality, and showed only limited and inefficient function to regenerate and form a cartilage surface after their transplantation.

Accordingly, a need exists for improved methods of regenerating cartilage.

### SUMMARY OF THE INVENTION

Applicants have investigated the potential of cartilage tissue isolated from polydactyly-derived excision fingers and toes during normal treatment practice in the US for polydactyly patients, which surgeries treat a higher age group of patients compared to Japan. Applicants identified that the chondrocytes isolated from the polydactyly-derived cartilage tissue are composed not only of mature chondrocyte cells but also chondroprogenitor cells (e.g., chondrocyte progenitor cells or chondroblasts) which contain transcription factors that promote differentiation into chondrocytes and are positive for immunostaining using an antibody against type II collagen. These chondroprogenitor cells show vigorous growth and the ability to form cell sheets and treat knee cartilage surface by generating hyaline cartilage structures. These cell sheets are expected to be both a functionally and economically competitive medical product. Applicants therefore identified an improved cartilage tissue cell source for the preparation of cell sheets. This disclosure describes preparation and properties of chondrocyte cell sheets that are produced from cells from this particular type of cartilage tissue, and their use for generating cartilage tissue in a subject. Mixtures of chondrocytes and chondroprogenitor cells were used to prepare cell sheets in vitro in temperature-responsive cell culture dishes (TRCDs) coated with a temperature-responsive polymer. Confluent cell sheets were detached from the TRCD by cooling the cultures to room temperature.

In certain aspects, the disclosure relates to a cell sheet for cartilage tissue repair, wherein the cell sheet is formed from cultured cells derived from cartilage tissue, wherein the cultured cells comprise chondrocytes and cells expressing transcription factors that promote differentiation into chondrocytes. In certain embodiments, the cultured cells further comprise cells containing cytokines and genes related to extracellular matrix that promote differentiation into chondrocytes.

In certain embodiments, at least 1% of the cultured cells express transcription factors that promote differentiation into chondrocytes. In certain embodiments, the cell sheet is positive for immunostaining using an antibody against type II collagen. In certain embodiments, the cell sheet spontaneously exhibits multiple layers of cells. In certain embodiments, the cell sheet is manually manipulated. In certain embodiments, the cartilage tissue is derived from polydactyly cartilage tissue of an animal. In certain embodiments, the species of animal is selected from the group consisting of: a human, a rabbit, a dog, a cat, a pig, a horse, a monkey, a chimpanzee, a rat, a mouse, a goat and a sheep. In certain embodiments, the cell sheet is used for treating a disease selected from the group consisting of: a cartilage partial defect, a cartilaginous injury and an osteochondral injury. In certain embodiments, the treating disease is by allogeneic or autologous transplantation.

In certain aspects, the disclosure relates to a method for producing a cell sheet for cartilage tissue repair, comprising:
(1) collecting cartilage tissue by scalpel in a clean environment, wherein the tissue includes cells containing transcription factors to differentiate into chondrocyte, from the part of cartilage tissue that appears black on an X-ray image,
(2) cutting the collected cartilage tissue with a scalpel into small pieces,
(3) collecting the cells through enzymatic treatment of the small pieces of cartilage tissue,
(4) primarily culturing the collected cells on the particular surface of material,
(5) thereafter, culturing the primarily cultured cells on a temperature responsive cell culture material, wherein a surface of the cell culture material is coated with a temperature responsive polymer which changes its hydration force within a temperature range between 0° C. and 80° C., in a temperature region wherein the polymer shows weak hydration force.
(6) detaching the cultured cells as the form of a cell sheet by adjusting the temperature of the surface of the temperature responsive cell culture material by lowering the temperature of its culture medium to a temperature at which the polymer shows a stronger hydration force.

In certain embodiments, the cartilage tissue is derived from the polydactyly tissue of animal. In certain embodiments, the particular surface of material is coated by any one type or combination of two or more types of the selected proteins from the group consisting of: a type I collagen, a laminin, a fibronectin and a Matrigel®. In certain embodiments, the surface of the temperature responsive material is coated/immobilized with block copolymer comprising a temperature responsive polymer and a hydrophobic polymer. In certain embodiments, an amount of the temperature responsive polymer in the block copolymer coated/immobilized on the surface of the temperature responsive material is within the range of 0.3 to 6.0 μg/cm². In certain embodiments, the temperature responsive polymer is poly(N-isopropyl acrylamide). In certain embodiments, the method of detaching the cell sheet from the culture surface is placing a carrier in intimate contact over the cell sheet at the end of culture process and detaching the cell sheet intact together with the carrier. In certain embodiments, the cell sheet comprises transgenic cells. In certain embodiments, the cell sheets are autonomously multi-layered, while the cells composing the cell sheet are proliferated. In certain embodiments, the cell sheet is artificially stacked on another cell sheets or layered onto another cell sheets repeatedly. In certain embodiments, the method of detaching the cell sheet is processed without treatment by a proteinase. In certain embodiments, the culture medium does not comprise serum.

In certain aspects, the disclosure relates to a chondrocyte cell sheet comprising one or more layers of confluent cells comprising chondrocytes and chondroprogenitor cells, wherein the cell sheet is prepared from a mixture of cells obtained from cartilage tissue of a subject, wherein the mixture of cells obtained from the cartilage tissue comprises chondrocytes and chondroprogenitor cells. In certain embodiments, the chondrocyte cell sheet exhibits increased expression of one or more genes selected from the group consisting of ACVR2B, ADAMTS12, BBS2, BMPR1B, COL2A1, COL9A2, COL11A1, COL11A2, COL12A1, ETS2, EXTL1, FBN2, FGFR3, HAS2, HMGA2, HOXA11, HOXA13, HOXD9, HOXD10, HOXD12, HOXD13, MEX3C, MMP13, MSX1, NAB2, NOG, RARA, RUNX2, RUNX3, SATB2, SIGLEC15, SIX1, SIX4, SOX6, SOX11, TGF-β1, TGF-β2, TIPARP, TRIM45, WNT5B, WNT7B, WNT11 and ZFAND5 relative to a cell sheet prepared from cells isolated from non-polydactyl cartilage from an adult.

In certain embodiments, the subject is human. In certain embodiments, the subject is less than 10 years old. In certain embodiments, the subject is 1.5 to 6 years old. In certain embodiments, the subject is selected from the group consisting of a rabbit, a dog, a cat, a pig, a horse, a monkey, a chimpanzee, a rat, a mouse, a goat and a sheep. In certain embodiments, the cartilage tissue of the subject is polydactyl cartilage tissue. In certain embodiments, at least 1% of the cells in the mixture of cells obtained from the cartilage tissue are chondroprogenitor cells. In certain embodiments, the chondrocytes and chondroprogenitor cells in the cell sheet express type I Collagen and type II Collagen. In certain embodiments, the chondrocytes and chondroprogenitor cells in the cell sheet express Transcription Factor for Cartilage (TFC). In certain embodiments, the chondrocyte cell sheet exhibits increased expression of one or more cytokines relative to a cell sheet prepared from cells isolated from non-polydactyl cartilage from an adult. In certain embodiments, the cytokines are selected from the group consisting of Transforming growth factor beta 1 (TGF-β1) and Transforming growth factor beta 2 (TGF-β2). In certain embodiments, the cell sheet consists essentially of chondrocytes and chondroprogenitor cells. In certain embodiments, the cell sheet comprises two or more layers of confluent cells comprising chondrocytes and chondroprogenitor cells. In certain embodiments, the cell sheet comprises three or more layers of confluent cells comprising chondrocytes and chondroprogenitor cells. In certain embodiments, at least 50% of cells in the cell sheet are chondrocytes.

In certain aspects, the disclosure relates to a composition comprising a chondrocyte cell sheet as described herein and a polymer-coated culture support that is removable from the cell sheet. In certain aspects, the disclosure relates to a composition comprising at least two of the chondrocyte cell sheets described herein. In certain embodiments, the at least two chondrocyte cell sheets are stacked on top of each other.

In certain aspects, the disclosure relates to a method for producing a chondrocyte cell sheet comprising one or more layers of confluent cells comprising chondrocytes and chondroprogenitor cells, the method comprising:
a) culturing a mixture of chondrocytes and chondroprogenitor cells in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the cell culture support, thereby producing a chondrocyte cell sheet comprising two or more layers of confluent cells comprising chondrocytes and chondroprogenitor cells.

In certain embodiments, the method further comprises:
d) collecting cartilage tissue by scalpel under sterile conditions, wherein the cartilage tissue comprises chondrocytes and chondroprogenitor cells;
e) cutting the collected cartilage tissue with scalpels into pieces; and
f) collecting cells from the cartilage tissue through enzymatic treatment of the pieces of cartilage tissue.

In certain embodiments, the adjusting step (b) is performed when the chondrocytes and chondroprogenitor cells are confluent. In certain embodiments, the culturing step (a) comprises adding the chondrocytes and chondroprogenitor cells to the culture solution at an initial cell density of at least 1×10⁵ cells/cm². In certain embodiments, the chondrocytes and chondroprogenitor cells are cultured in the culture solution on the temperature-responsive polymer for at least 2 days before the adjusting step (b). In certain embodiments, the cartilage tissue is derived from polydactyl cartilage tissue. In certain embodiments, the substrate surface of the cell culture support is coated with one or more proteins selected from the group consisting of: type I collagen, laminin, fibronectin, nidogen and heparan sulfate proteoglycan. In certain embodiments, the substrate surface of the cell culture support is coated with block copolymer comprising a temperature responsive polymer and a hydrophobic polymer. In certain embodiments, the temperature responsive polymer is coated on the substrate surface at a concentration within the range of 0.3 to 6.0 μg/cm². In certain embodiments, the temperature responsive polymer is poly(N-isopropyl acrylamide). In certain embodiments, the detaching step c) comprises placing a carrier in contact with the cell sheet and detaching the cell sheet intact together with the carrier. In certain embodiments, the detaching step c) does not comprise treatment with a proteinase. In certain embodiments, the culture solution does not comprise serum. In certain embodiments, the cell sheet comprises transgenic cells. In certain embodiments, the chondrocyte cell sheet comprises two or more layers of confluent cells comprising chondrocytes and chondroprogenitor cells. In certain embodiments, the chondrocyte cell sheet comprises three or more layers of confluent cells comprising chondrocytes and chondroprogenitor cells. In certain embodiments, the method further comprises stacking at least two cell sheets on top of each other. In certain aspects, the disclosure relates to a cell sheet produced by a method described herein.

In certain aspects, the disclosure relates to a method of generating cartilage tissue in a subject, the method comprising applying a chondrocyte cell sheet as described herein, or a composition as described herein, to cartilage tissue in a subject. In certain aspects, the disclosure relates to a method of treating a disorder in a subject, the method comprising applying a chondrocyte cell sheet as described herein, or a composition as described herein to cartilage tissue in a subject. In certain embodiments, the disorder is selected from the group consisting of: a cartilage partial defect, a cartilaginous injury and an osteochondral injury. In certain embodiments, the chondrocyte cell sheet regenerates hyaline cartilage tissue when the cell sheet is applied to the cartilage tissue in the subject.

In certain aspects the disclosure relates to a cell sheet for cartilage tissue repair, wherein the cell sheet being formed of a preparation of cultured cell group derived from the cartilage tissue, wherein the cultured cell group comprises chondrocyte and the cells having/containing transcription factors to differentiate into chondrocyte. In certain embodiments, a part of the cultured cell group is composed by the cells having/containing the factors/(such as cytokines, as well as genes related to extracellular matrix) and which helps differentiation into chondrocyte. In certain embodiments, the cultured cell group, at least 1% of it, is composed by the cells having/containing transcription factors to differentiate into chondrocyte. In certain embodiments, the cell sheet is positive for immunostaining using an antibody against type II collagen. In certain embodiments, the cell sheets are spontaneously/autonomously multilayered. In certain embodiments, the cell sheets being artificially stacked/multilayered. In certain embodiments, the cartilage tissue is derived from the polydactyly cartilage tissue of animal. In certain embodiments, the species of animal are selected from the group consisting of: a human, a rabbit, a dog, a cat, a pig, a horse, a monkey, a chimpanzee, a rat, a mouse, a goat and a sheep.

In certain embodiments, the cell sheet are used for treating disease selected from the group of diseases consisting of: a cartilage partially defect, a cartilaginous injury and an osteochondral injury. In certain embodiments, the treating disease is by allogeneic or autologous transplantation.

In certain aspects the disclosure relates to a method for producing a cell sheet for cartilage tissue repair, comprising characteristic steps of:
(1) collecting the cartilage tissue by scalpels in a clean environment, which tissue includes the cells containing transcription factors to differentiate into chondrocyte, from the part of cartilage tissue that appears black on an X-ray image,
(2) cutting the collected cartilage tissue with scalpels into small pieces/,
(3) collecting the cells through enzymatic treatment of the small pieces/parts of cartilage tissue,
(4) primarily culturing the collected cells on the particular surface of material,
(5) thereafter, culturing the primarily cultured cells on a temperature responsive cell culture materials, which surface is coated with a temperature responsive polymer which changes its hydration force within a temperature range between 0° C. and 80° C., in a temperature region wherein the polymer shows weak hydration force.
(6) detaching the cultured cells as the form of cell sheet, by adjusting the temperature of the surface of temperature responsive cell culture material by lowering temperature of its culture medium to a temperature at which the polymer shows a stronger hydration force.

In certain embodiments, the cartilage tissue is derived from the polydactyly tissue of animal. In certain embodiments, the particular surface of material is coated by any one type or combination of two or more types of the selected proteins from the group consisting of: a type I collagen, a laminin, a fibronectin and a Matrigel®. In certain embodiments, the surface of the temperature responsive material is coated/immobilized with block copolymer comprising a temperature responsive polymer and a hydrophobic polymer. In certain embodiments, an amount of the temperature responsive polymer in the block copolymer coated/immobilized on the surface of the temperature responsive material is within the range of 0.3 to 6.0 μg/cm². In certain embodiments, the temperature responsive polymer is poly(N-isopropyl acrylamide).

In certain embodiments, the method of detaching the cell sheet from the culture surface is placing a carrier in intimate contact over the cell sheet at the end of culture process and detaching the cell sheet intact together with the carrier. In certain embodiments, the cell sheet comprises transgenic cells. In certain embodiments, the cell sheets are autonomously multi-layered, while the cells composing the cell sheet are proliferated. In certain embodiments, the cell sheet is artificially stacked on another cell sheets or layered onto another cell sheets repeatedly. In certain embodiments, the method of detaching the cell sheet is processed without treatment by a proteinase. In certain embodiments, the culture medium does not involve a human serum.

In certain aspects the disclosure relates to a chondrocyte cell sheet comprising one or more layers of confluent chondrocytes prepared from a mixture of cells obtained from cartilage tissue of a subject, wherein the cells obtained from the cartilage tissue comprise chondrocytes and cells that will differentiate into chondrocytes. In certain embodiments, the subject is human. In certain embodiments, the subject is 1.5 to 6 years old. In certain embodiments, the cartilage tissue is polydactyl cartilage tissue. In certain embodiments, the chondrocytes in the cell sheet produce Collagen type II. In certain embodiments, the chondrocytes in the cell sheet express Transcription Factor for Cartilage (TFC). In certain embodiments, the chondrocytes express cytokines. In certain embodiments, the cell sheet consists essentially of chondrocytes. In certain embodiments, the cell sheet comprises more than one layer of chondrocytes. In certain embodiments, at least 50% of cells in the cell sheet are chondrocytes. In certain embodiments, the subject is selected from the group consisting of a rabbit, a dog, a cat, a pig, a horse, a monkey, a chimpanzee, a rat, a mouse, a goat and a sheep.

In certain aspects the disclosure relates to a composition comprising a cell sheet as described herein and a polymer-coated culture support that is removable from the cell sheet. In certain aspects the disclosure relates to a composition comprising at least two of the cell sheets described herein. In certain embodiments, the at least two cell sheets are stacked on top of each other.

In certain aspects the disclosure relates to a method for producing a chondrocyte cell sheet comprising one or more layers of confluent chondrocytes, the method comprising:
a) culturing chondrocytes in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the culture support.

In certain embodiments, the method further comprises:
d) collecting cartilage tissue by scalpel under sterile conditions, wherein the cartilage tissue comprises cells containing transcription factors to differentiate into chondrocytes; e) cutting the collected cartilage tissue with scalpels into pieces; and
f) collecting cells from the cartilage tissue through enzymatic treatment of the pieces of cartilage tissue.

In certain embodiments, the adjusting step (b) is performed when the chondrocytes are confluent. In certain embodiments, the culturing step (a) comprises adding the chondrocytes to the culture solution at an initial cell density of at least 1×10⁵ cells/cm². In certain embodiments, the chondrocytes are cultured in the culture solution on the temperature-responsive polymer for at least 8 days before the adjusting step (b). In certain embodiments, the cartilage tissue is derived from polydactyl cartilage tissue. In certain embodiments, the substrate surface of the cell culture support is coated with a protein selected from the group consisting of: type I collagen, laminin, fibronectin, nidogen and heparan sulfate proteoglycan. In certain embodiments, the substrate surface of the cell culture support is coated with block copolymer comprising a temperature responsive polymer and a hydrophobic polymer.

In certain embodiments, the temperature responsive polymer is coated on the substrate surface at a concentration within the range of 0.3 to 6.0 μg/cm². In certain embodiments, the temperature responsive polymer is poly(N-isopropyl acrylamide). In certain embodiments, the detaching step c) comprises placing a carrier in contact with the cell sheet and detaching the cell sheet intact together with the carrier. In certain embodiments, the detaching step c) does not comprise treatment with a proteinase. In certain embodiments, the culture solution does not comprise human serum. In certain embodiments, the cell sheet further comprises transgenic cells.

In certain embodiments, the chondrocyte cell sheet comprises more than one layer of confluent chondrocytes. In certain embodiments, the method further comprises stacking at least two cell sheets on top of each other. In certain aspects the disclosure relates to a cell sheet produced by the methods described herein.

In certain aspects the disclosure relates to a method of generating cartilage tissue in a subject, the method comprising applying a cell sheet as described herein or a composition as described herein to cartilage tissue in a subject.

In certain aspects the disclosure relates to a method of treating a disorder in a subject, the method comprising applying a cell sheet as described herein or a composition as described herein to cartilage tissue in a subject. In certain embodiments, the disorder is selected from the group consisting of: a cartilage partial defect, a cartilaginous injury and an osteochondral injury. In certain embodiments, the chondrocytes are autologous to the subject. In certain embodiments, the chondrocytes are allogeneic to the subject. In certain embodiments, the subject is human.

### BRIEF DESCRIPTION OF THE DRAWINGS

FIGS. 1A, 1B and 1C show stable growth of polydactyly derived chondrocytes (PDC). Polydactyly-derived chondrocytes (PDC): n=10; Normal adult chondrocytes (NAC): n=6.

FIGS. 2A and 2B show safranin O staining of chondrocyte cell sheets. A thicker cell sheet was obtained with polydactyly-derived cells (PDCS) compared to normal adult-derived cells (NACS). All cell sheets were fabricated at passage 2 (P2) in the same culture condition. Bars: 50 μm.

FIG. 3 shows that a cell sheet prepared from polydactyly-derived cells (PDCS) contained more cells than a cell sheet prepared from normal adult-derived cells (NACS). Plots represent the total cell number in one cell sheet from individual donors. Data shown as mean and SD.

FIG. 4 shows immunohistochemistry of type collagen 2. Type 2 collagen was detected by using mouse monoclonal antibody (clone 2B1.5) whereas isotype control did not show any color development. Hematoxylin was used as a counter staining.

FIG. 5 shows gene ontology term enrichment in polydactyly-derived cells (PDCS) vs. normal adult-derived cells (NACS). RNAseq data from n=7 (PDCS) and n=5 (NACS). RNA was extracted from cell sheets using RNA Mini kit (Qiagen) (PDCS; n=7, NACS; n=5). 1 ug of RNA was reverse transcribed and the cDNA samples were used for pair-end sequencing using Illumina next generation sequencer, HiSeq. The quantification data was normalized, then gene ontology (GO) enrichment was analyzed with GOseq software. Statistically most significant GOs are listed in FIG. 5.

FIG. 6 shows a list of upregulated genes of the significantly different gene ontologies of interest.

FIG. 7 shows hyaline cartilage regeneration by polydactyly-derived cells (PDCS) in a rat focal defect model.

FIG. 8 shows rapid recovery of weight distribution ratio by polydactyly-derived cells (PDCS) transplantation. Sample numbers at week 2, 3, 4, 5, and 6=13, 12, 12, 8, and 8 (defect only group); =16, 15, 15, 9, and 9 (defect+CS group), respectively. Data shown as mean and SEM. **p<0.01, *p<0.05 by Welch's t-test.

FIGS. 9A-9E show isolation of chondrocytes from juvenile cartilage surgical discards and in vitro expansion of juvenile chondrocytes. FIG. 9A shows juvenile donor-derived cartilage tissues under stereo microscope. Scale bar: 5 mm. FIG. 9B shows Safranin O staining of cartilage tissue. Scale bar: 500 m. FIG. 9C shows phase contrast images of cultured chondrocytes. Scale bars: 200 m. FIG. 9D shows average fold-change of in vitro cell expansion. Data shown as mean and SD (n=13 individual donors). FIG. 9E shows in vitro differentiated of JCC pellets. Photos show Safranin-O staining of P2 JCCs (top) and P9 JCCs (bottom). Bars: 500 10 μm. Right graph shows pellet size diameter measurements (n=2). Error bars indicate SD. **p<0.01 by Student's t-test.

FIGS. 10A-10F show characterization of engineered JCC sheet. (A) A phase contrast image of confluent chondrocytes at day 14 of passage 2. Scale bar: 200 μm. (B) Macroscopic image of engineered JCC sheet. Scale bar: 5 mm. (C) Total cell number in one cell sheet (n=13 individual donors). (D) Hematoxylin and eosin staining, Safranin-O staining, Toluidine blue staining, aggrecan, type I collagen, and type II collagen immunohistochemistry of JCC sheets. Bars: 50 μm. (E) Safranin-O staining of an in vitro differentiated pellet from P2 JCC sheet. Bar: 500 μm. (F) Theoretical cell sheet numbers possibly prepared from cultured JCCs at each passage. Data shown as mean and SD (n=11 individual donors).

FIGS. 11A-11C show tumorigenicity assay, population doubling time, and cell surface markers. (A) (a) Microscopic images of in vitro tumorigenicity assay in soft agar culture conditions. Images of left column show seeded cells from 2-week cultured cell sheet. Images of middle column show seeded cells from 3.5-week cultured cell sheet. Images of right column show seeded HepG2 cells as positive control. Top row shows Preclinical Safety and Efficacy of Juvenile Chondrocyte Sheets the image at day 0 and bottom shows images at day 8. Bars: 200 μm. (b) Semi-quantification of cell number by DNA-bound fluorescence in soft agar culture at day 0 and day 8. Data shown as mean and SD (n=4 individual donors). **p<0.01, *p<0.05, N.S. (non-significant) by Student't-test. (B) Flow cytometry analysis for cell purity and surface marker characterization. (a) Representative histograms for CD45, lineage cocktail (mixture of CD3, CD14, CD16, CD19, CD20, CD56), CD31, HLA-ABC and HLA-DR, -DP, -DQ, CD44, CD90, CD81, and CD106. Column colors represent fluorophores (blue: Pacific blue, green: FITC or Alx488, red: PE, magenta: APC or Alx647) (b) Average percentages for CD45, lineage cocktail (mixture of CD3, CD14, CD16, CD19, CD20, CD56), CD31, HLA-ABC and HLA-DR, -DP, -DQ, CD44, CD90, CD81, and CD106. n=4-6 individual donors. (C) Population doubling time in the extended subculture in chondrocyte culture medium up to P13. Data shown as mean and SD (n=13 individual donors).

FIGS. 12A and 12B show middle- and long-term in vivo efficacy of focal osteochondral defect treatment in nude rats. (A) Macroscopic images of surgically created focal defects (left images of each group) and 4, 8, 12, and 24 weeks after treatments (right images of each time point). Top row shows non-treatment group. Bottom row shows defect and cell sheet group. (B) Safranin-O staining of each condition. Representative images of samples at 4 weeks (n=14), 8 weeks (n=3), 12 weeks (n=3), 24 weeks (n=3) are shown. Bars: Top and third rows: 500 μm, second and bottom rows: 100 μm.

FIG. 13 shows cartilage-specific marker expression by transplanted human JCC sheet-derived tissue. Aggrecan staining (left), type II collagen staining (center), type I collagen staining (right) are shown. All samples shown are 4 weeks after transplantation. Bars: Left columns of each group: 500 μm, right columns of each group: 100 μm.

FIGS. 14A and 14B show transplanted human chondrocyte engraftment accompanied by type II collagen deposition. (A) (a) Human antigen-specific vimentin staining of a JCC sheet-treated sample. Red arrowheads indicate regenerated cartilage. Blue arrowheads indicate host cartilage. Bar: 500 μm (b) Magnified area of regenerated cartilage with human antigen specific vimentin staining. Bar: 200 μm. (B) Double staining of human vimentin (red) and type II collagen (green). DAPI+Ph: DAPI+phase contrast image to show nuclei (blue with white edge). Right panel shows the merged image of human vimentin, type II collagen, and DAPI. Bars: 200 μm. A histology sample of 4 weeks is shown.

FIG. 15 shows human vimentin-specific staining of rat knee 24-weeks after the transplantation of a polydactyly-derived chondrocyte cell sheet. The darker areas indicate the human vimentin.

FIG. 16 shows immunohistochemistry of knee samples 24-weeks after cell sheet transplantation. Proteins shown are type I collagen (COL1), type II collagen (COL2) and aggrecan (ACAN). The darker areas indicate the positive area for each protein.

### DETAILED DESCRIPTION OF THE INVENTION

Applicants have investigated, as an allogeneic chondrocyte cell source, the potential of cartilage tissue isolated from polydactyly-derived excision fingers and toes during normal treatment practice in the US for polydactyly patients which surgeries treat higher age group of patients compared to Japan. Applicants identified that the cell sheets which were positive for immunostaining using an antibody against type II collagen and positive against type I collagen were quite different from the above two technologies described in US2008/0226692 and US2018/0243476, since the cells sheets were produced in large numbers immediately and maintained constant quality. It appears that the cells isolated from the polydactyly-derived cartilage tissue are composed not only of mature chondrocyte cells, but also chondroprogenitor cells which contain transcription factors (e.g., one or more of HOXA11, RUNX3, SOX6, RUNX2, HOXD9, HOXD10, and HOXD3) that promote differentiation into chondrocytes. These cells show vigorous growth and effective function to form cell sheets uniformly, densely and immediately in large numbers and treat knee cartilage surface by generating hyaline structures. The cell sheets were positive for immunostaining using an antibody against type II collagen and positive against type I collagen. Cell sheets generated from these aggregated cells are expected to be not only a functionally but also economically competitive medical product to provide the most effective and sufficient treatment in a timely manner and maintain quality of life of patients by solving the issue of cell source volume limitation as well as time gap.

This disclosure describes preparation and properties of chondrocyte cell sheets which are produced from the cells in the specific cartilage tissue in the living body and their use for generating cartilage tissue in a subject. Chondrocytes were used to prepare cell sheets in vitro in temperature-responsive cell culture dishes (TRCDs) coated with a temperature-responsive polymer. Confluent cell sheets were detached from the TRCD by cooling the cultures to room temperature. In addition, application of the chondrocyte cell sheets to cartilage in a rat focal defect model demonstrated hyaline cartilage regeneration.

### Definitions

The term “chondrocyte cell sheet” as used herein refers to a cell sheet obtained by growing chondrocytes and chondroprogenitor cells on a cell culture support in vitro.

The term “chondroprogenitor cells” as used herein refers to a population of stem/progenitor cells that are capable of differentiating into chondrocytes. Chondroprogenitor cells include, but are not limited to, chondrocyte progenitor cells, chondroblasts, cartilage progenitor cells, and bone marrow mesenchymal stem cells. Chondroprogenitor cells exhibit different characteristics from mature chondrocytes including high affinity for fibronectin, high colony-forming efficiency, and expression of the Notch1 gene. See Jayasuriya et al., 2016, Connect Tissue Res. 56(4): 265-271, which is incorporated by reference herein in its entirety.

### I. Chondrocytes and Chondroprogenitor Cells for Use in Preparing Chondrocyte Cell Sheets

This disclosure provides a cultured chondrocyte cell sheet used for cartilage tissue repair. In this disclosure, the cells used to prepare the chondrocyte cell sheet comprise chondrocytes and chondroprogenitor cells. In some embodiments, the chondroprogenitor cells comprise one or more transcription factors selected from the group consisting of HOXA11, RUNX3, SOX6, RUNX2, HOXD9, HOXD10, and HOXD3. In some embodiments, the cartilage tissues used as a source of cells for producing the cultured chondrocyte cell is a cartilage tissue that functions under mechanical load. In some embodiments, the source of the cells for preparation of the cell sheets is excess cartilage tissue from excision of fingers or toes in the treatment of polydactyly. Cells isolated from surplus cartilage tissue of polydactyly after finger resection are more proliferative than cells isolated from normal adult cartilage tissues. As a result, many chondrocyte cell sheets may be obtained from one individual's polydactyly cartilage. The cells isolated from polydactyly tissue contain both mature chondrocytes and chondroprogenitor cells. The chondroprogenitor cells comprise transcription factors (e.g., one or more of HOXA11, RUNX3, SOX6, RUNX2, HOXD9, HOXD10, and HOXD3) that promote differentiation into chondrocytes. Applicant has demonstrated that chondrocyte cell sheets prepared from a mixture of chondrocytes and chondroprogenitor cells adhere to the transplantation site well, and show a cartilage tissue regeneration effect.

In some embodiments, at least 1%, at least 2%, at least 3%, at least 4%, at least 5%, at least 10%, at least 15%, at least 20%, at least 25%, at least 30%, at least 40% or at least 50% of the cells used to prepare the chondrocyte cell sheets are chondroprogenitor cells. In a particular embodiment, at least 1% of the cells used to prepare the chondrocyte cell sheets Suitable chondroprogenitor cells include, but are not limited to, chondroblasts, cartilage progenitor cells, chondrocyte progenitor cells, bone marrow mesenchymal stem cells, and mixtures thereof. In some embodiments, at least 1%, at least 2%, at least 3%, at least 4%, at least 5%, at least 10%, at least 15%, at least 20%, at least 25%, at least 30%, at least 40% or at least 50% of the cells used to prepare the chondrocyte cell sheets are chondrocytes.

The cells used to prepare the chondrocyte cell sheets described herein may contain cells other than chondrocytes and chondroprogenitor cells, including but not limited to epithelial cells, epithelial stem cells, fibroblasts, vascular endothelial cells, vascular endothelial progenitor cells, bone marrow-derived cells, fat-derived cells, mesenchymal stem cells, or any combination thereof. Also, it is not particularly limited for the respective content ratio of those cells.

As described above, in some embodiments the cells used to prepare the chondrocyte cell sheets described herein comprise chondroprogenitor cells comprising transcription factors that promote differentiation into chondrocytes (e.g., one or more of HOXA11, RUNX3, SOX6, RUNX2, HOXD9, HOXD10, and HOXD3). These chondroprogenitor cells may also contain one or more factors that help cartilage regeneration, including but not limited to BMP-bone morphogenetic protein, IGF-insulin-like growth factor, TGF-β-transforming growth factor-beta, cartilage derived matrix protein-1, SOX-sry-related HMG box) 5,6,9, PTH (parathyroid hormone), and its related proteins, and hedgehog family proteins, e.g., Sonic hedgehog (SHH); Indian hedgehog (IHH); and Desert hedgehog (DHH).

In some embodiments, the chondrocytes and/or chondropogenitor cells used to prepare the chondrocyte cell sheets contain type II collagen. For example, in some embodiments, the cells used to prepare the chondrocyte cell sheets are positive when stained with an antibody against type II collagen.

In some embodiments, the cells used to prepare the chondrocyte cell sheets are isolated directly from living tissue, e.g., cartilage tissue. In some embodiments, cells are collected from living tissue, e.g., cartilage tissue, and then cultured in vitro before preparing the chondrocyte cell sheets. In some embodiments, the cells used to prepare the chondrocyte cell sheets are isolated from a mammal. In some embodiments, the cells used to prepare the chondrocyte cell sheets are isolated from a subject selected from the group consisting of a human, a rat, a mouse, a guinea pig, a marmoset, a rabbit, a dog, a cat, a sheep, a pig, a horse, a rat, a mouse, a goat, a monkey, and a chimpanzee. In a particular embodiment, the cells used to prepare the chondrocyte cell sheets are isolated from a human. In a particular embodiment, the cells used to prepare the chondrocyte cell sheets are isolated from a chimpanzee. For example, when using chondrocyte cell sheets in the treatment of humans, pigs, or monkeys, cells isolated from chimpanzees may be used. In some embodiments, the cells used to prepare the chondrocyte cell sheets are isolated from an immunodeficient subject. In some embodiments, the subject (e.g., a human subject) is polydactyl. In some embodiments, the chondrocytes are obtained from polydactyl cartilage tissue (e.g., human polydactyl cartilage tissue).

In a particular embodiment, the cells used to prepare the chondrocyte cell sheets are isolated from a human. In some embodiments, the human is 1.5, 2, 2.5, 3, 3.5, 4, 4.5, 5, 5.5, 6, 7, 8, 9 or 10 years old. In some embodiments, the human is at least 1.5, 2, 2.5, 3, 3.5, 4, 4.5, 5, 5.5, 6, 7, 8, 9 or 10 years old. In some embodiments, the human in less than 1.5, 2, 2.5, 3, 3.5, 4, 4.5, 5, 5.5, 6, 7, 8, 9 or 10 years old. Any of these values may be used to define the age of the subject from which the chondrocytes are isolated. For example, in some embodiments, the subject is less than 10 years old, 1.5-6 years old, or 2-5 years old. In a particular embodiment, the subject is 1.5 to 6 years old.

Any suitable methods may be used to isolate the cells from cartilage tissue, e.g., polydactyly cartilage tissue. Chondrocytes and chondroprogenitor cells may be isolated from cartilage tissue, (e.g., cartilage tissue that appears black on an X-ray image) by collecting cartilage tissue by scalpel under sterile conditions, cutting the collected cartilage tissue with scalpels into pieces, and isolating cells from the cartilage tissue through enzymatic treatment of the pieces of cartilage tissue. An explant culture method may also be used by placing the cartilage tissue layer on a culture substrate. For example, for the explant culture method, the cartilage is chopped into pieces (e.g., pieces about 1 mm in diameter or less) and placed on a culture surface with a small amount of culture medium to facilitate the cell outgrowth. When the cells reach confluence or sub-confluence on the culture surface, the cells are collected. These methods may also be combined.

Any suitable culture medium may be used to culture the cells for preparation of the chondrocyte cell sheet. Suitable culture media include, but are not limited to, F-12 medium, DMEM medium, or a mixture thereof. In some embodiments, serum (e.g., human serum, fetal bovine serum (FBS) or fetal calf serum (FCS)) may be added to the culture medium. For example, Applicants have demonstrated that by adding human serum to the culture medium, the cells obtain greater physical strength, and as a result, the cultured human chondrocytes sheets achieve improved flexibility. In some embodiments, the culture medium does not comprise serum, e.g., human serum, fetal bovine serum (FBS) or fetal calf serum (FCS).

In some embodiments, the concentration of the serum (e.g., human serum) in the medium ranges from 0.5% to 35%, from 1% to 30%, from 5% to 25%, or from 10% to 20%, for culturing the cells. In a particular embodiment, the concentration of the serum in the medium is 20%.

II. Chondrocyte Cell Sheets Produced from Chondrocytes and Chondroprogenitor Cells

In certain aspects the present disclosure relates to a chondrocyte cell sheet comprising one or more layers of confluent chondrocytes. The term “chondrocyte cell sheet” as used herein refers to a cell sheet obtained by growing chondrocytes and chondroprogenitor cells on a cell culture support in vitro. The chondrocyte sheets described herein are harvested as a single sheet with a temperature shift using a temperature-responsive culture dish (TRCD) without any enzyme treatment. Accordingly, in certain aspects, the present disclosure relates to a composition comprising a chondrocyte cell sheet as described herein and a polymer-coated culture support (e.g., a culture dish) that is removable from the cell sheet.

In certain aspects, the present disclosure relates to a chondrocyte cell sheet comprising two or more layers of confluent cells comprising chondrocytes and chondroprogenitor cells, wherein the cell sheet is prepared from a mixture of cells obtained from cartilage tissue of a subject, wherein the mixture of cells obtained from the cartilage tissue comprises chondrocytes and chondroprogenitor cells.

In some embodiments, the chondrocyte cell sheet exhibits increased expression of one or more genes selected from the group consisting of ACVR2B, ADAMTS12, BBS2, BMPR1B, COL2A1, COL9A2, COL11A1, COL11A2, COL12A1, ETS2, EXTL1, FBN2, FGFR3, HAS2, HMGA2, HOXA11, HOXA13, HOXD9, HOXD10, HOXD12, HOXD13, MEX3C, MMP13, MSX1, NAB2, NOG, RARA, RUNX2, RUNX3, SATB2, SIGLEC15, SIX1, SIX4, SOX6, SOX11, TGFB1, TGFB2, TIPARP, TRIM45, WNT5B, WNT7B, WNT11 and ZFAND5 relative to a cell sheet prepared from cells isolated from non-polydactyl cartilage from an adult. In some embodiments, the chondrocyte cell sheet exhibits increased expression of one or more transcription factors selected from the group consisting of HOXA11, RUNX3, SOX6, RUNX2, HOXD9, HOXD10, and HOXD3 relative to a cell sheet prepared from cells isolated from non-polydactyl cartilage from an adult. In some embodiments, the chondrocyte cell sheet exhibits a 50%, 100%, 150%, 200%, 250%, 300%, 400% or 500% increase in expression of one or more genes selected from the group consisting of ACVR2B, ADAMTS12, BBS2, BMPR1B, COL2A1, COL9A2, COL11A1, COL11A2, COL12A1, ETS2, EXTL1, FBN2, FGFR3, HAS2, HMGA2, HOXA11, HOXA13, HOXD9, HOXD10, HOXD12, HOXD13, MEX3C, MMP13, MSX1, NAB2, NOG, RARA, RUNX2, RUNX3, SATB2, SIGLEC15, SIX1, SIX4, SOX6, SOX11, TGFB1, TGFB2, TIPARP, TRIM45, WNT5B, WNT7B, WNT11 and ZFAND5 relative to a cell sheet prepared from cells isolated from non-polydactyl cartilage from an adult. In some embodiments, the chondrocyte cell sheet exhibits a 50%, 100%, 150%, 200%, 250%, 300%, 400% or 500% increase in expression of one or more genes selected from the group consisting of HOXA11, RUNX3, SOX6, RUNX2, HOXD9, HOXD10, and HOXD3 relative to a cell sheet prepared from cells isolated from non-polydactyl cartilage from an adult. In some embodiments, the expression of TGFB1 is increased in the chondrocyte cell sheet by at least 150% relative to a cell sheet prepared from cells isolated from non-polydactyl cartilage from an adult. In some embodiments, the expression of TGFB2 is increased in the chondrocyte cell sheet by at least 250% relative to a cell sheet prepared from cells isolated from non-polydactyl cartilage from an adult.

In some embodiments, the chondrocytes and chondroprogenitor cells in the chondrocyte cell sheet produce type II collagen. For example, in some embodiments, the cells within the chondrocyte cell sheet are positive when stained with an antibody against type II collagen. In some embodiments, the chondrocytes and chondroprogenitor cells in the chondrocyte cell sheet produce type I collagen. In some embodiments, the chondrocytes and chondroprogenitor cells in the chondrocyte cell sheet produce type I collagen and type II collagen. In some embodiments, the chondrocytes and chondroprogenitor cells in the chondrocyte cell sheet express Transcription Factor for Cartilage (TFC). In some embodiments, the chondroprogenitor cells in the chondrocyte cell sheet express one or more transcription factors selected from the group consisting of HOXA11, RUNX3, SOX6, RUNX2, HOXD9, HOXD10, and HOXD3. In some embodiments, the chondroprogenitor cells in the chondrocyte cell sheet comprise one or more factors that help cartilage regeneration, including but not limited to BMP-bone morphogenetic protein, IGF-insulin-like growth factor, TGF-β-transforming growth factor-beta, cartilage derived matrix protein-1, SOX-sry-related HMG box) 5,6,9, PTH (parathyroid hormone), and its related proteins, and hedgehog family proteins, e.g., Sonic hedgehog (SHH); Indian hedgehog (IHH); and Desert hedgehog (DHH).

In some embodiments, at least 10%, 20%, 30%, 40%, 50%, 60%, 70%, 80%, 90%, 95% or 99% of cells in the chondroctye cell sheet are chondrocytes. In some embodiments, the cell sheet consists of or consists essentially of chondrocytes. In some embodiments, at least 1%, at least 2%, at least 3%, at least 4%, at least 5%, at least 10%, at least 15%, at least 20%, at least 25%, at least 30%, at least 40% or at least 50% of the cells in the chondrocyte cell sheet are chondroprogenitor cells. In a particular embodiment, at least 1% of the cells in the chondrocyte cell sheet are chondroprogenitor cells.

### III. Methods for Producing Chondrocyte Cell Sheets In Vitro

In certain aspects, the present disclosure relates to a method for producing a chondrocyte cell sheet comprising one or more layers of confluent chondrocytes, the method comprising:
(1) collecting the cartilage tissue by scalpels in a clean environment, which tissue includes the cells containing transcription factors to differentiate into chondrocyte, from the part of cartilage tissue that appears black on an X-ray image,
(2) cutting the collected cartilage tissue with scalpels into small pieces,
(3) collecting the cells through enzymatic treatment of the small pieces/parts of cartilage tissue,
(4) primarily culturing the collected cells on the particular surface of material,
(5) thereafter, culturing the primarily cultured cells on a temperature responsive cell culture materials, which surface is coated with a temperature responsive polymer which changes its hydration force within a temperature range between 0° C. and 80° C., in a temperature region wherein the polymer shows weak hydration force.
(6) detaching the cultured cells as the form of cell sheet, by adjusting the temperature of the surface of temperature responsive cell culture material by lowering temperature of its culture medium to a temperature at which the polymer shows a stronger hydration force.

In certain aspects, the present disclosure relates to a method for producing a chondrocyte cell sheet comprising two or more layers of confluent cells comprising chondrocytes and chondroprogenitor cells, the method comprising:
a) culturing a mixture of chondrocytes and chondroprogenitor cells in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the cell culture support, thereby producing a chondrocyte cell sheet comprising two or more layers of confluent cells comprising chondrocytes and chondroprogenitor cells.

In some embodiments, the step of adjusting the temperature of the culture solution to release the cell sheet from the culture support is performed when the chondrocytes and chondroprogenitor cells are confluent.

In some embodiments, the detaching step (6) comprises placing a carrier in contact with the cell sheet and detaching the cell sheet intact together with the carrier. In some embodiments, the detaching step (6) does not comprise treatment with a proteinase. In some embodiments, the cell sheet further comprises transgenic cells.

General methods for preparing cell sheets are known in the art and are described, for example, in U.S. Pat. Nos. 8,642,338; 8,889,417; 9,981,064; and 9,114,192, each of which is incorporated by reference herein in its entirety.

The temperature-responsive polymer used to coat the substrate of the cell culture support has an upper or lower critical solution temperature in aqueous solution which is generally in the range of 0° C. to 80° C., for example, 10° C. to 50° C., 15° C. to 40° C., or 20° C. to 35° C.

The temperature-responsive polymer may be a homopolymer or a copolymer. Exemplary polymers are described, for example, in Japanese Patent Laid-Open No. 211865/1990. Specifically, they may be obtained by homo- or co-polymerization of monomers such as, for example, (meth)acrylamide compounds ((meth)acrylamide refers to both acrylamide and methacrylamide), N-(or N,N-di)alkyl-substituted (meth)acrylamide derivatives, and vinyl ether derivatives. In the case of copolymers, any two or more monomers, such as the monomers described above, may be employed. Further, those monomers may be copolymerized with other monomers, one polymer may be grafted to another, two polymers may be copolymerized, or a mixture of polymer and copolymer may be employed. If desired, polymers may be crosslinked to an extent that will not impair their inherent properties.

In some embodiments, a surface of the cell culture support is coated with block polymer comprising a temperature responsive polymer and a hydrophobic polymer. In some embodiments, the temperature responsive polymer is coated on the substrate surface at a concentration within the range of 0.3 to 6.0 μg/cm². The surface of the cell culture support which is coated with the polymer may be of any type, including those which are commonly used in cell culture, such as glass, modified glass, polystyrene, poly(methyl methacrylate), and ceramics.

Methods of coating the support with the temperature-responsive polymer are known in the art and are described, for example, in Japanese Patent Laid-Open No. 211865/1990. Specifically, such coating can be achieved by subjecting the substrate and the above-mentioned monomer or polymer to, for example, electron beam (EB) exposure, irradiation with 7-rays, irradiation with UV rays, plasma treatment, corona treatment, or organic polymerization reaction. Other techniques such as physical adsorption as achieved by coating application and kneading may also be used. The coverage of the temperature responsive polymer may be in the range of 0.3-6.0 μg/cm², for example, 0.7-3.5 μg/cm², or 0.9-2.5 μg/cm². The morphology of the cell culture support may be, for example, a dish, a multi-plate, a flask, or a cell insert.

The cultured cells may be detached and recovered from the cell culture support by adjusting the temperature of the support material to the temperature at which the polymer on the support substrate hydrates, whereupon the cells can be detached. Smooth detachment can be realized by applying a water stream to the gap between the cell sheet and the support. Detachment of the cell sheet may be affected within the culture solution in which the cells have been cultivated or in other isotonic fluids, whichever is suitable.

In a particular embodiment, the temperature-responsive polymer is poly(N-isopropyl acrylamide). Poly(N-isopropyl acrylamide) has a lower critical solution temperature in water of 31° C. If it is in a free state, it undergoes dehydration in water at temperatures above 31° C. and the polymer chains aggregate to cause turbidity. Conversely, at temperatures of 31° C. and below, the polymer chains hydrate to become dissolved in water, thereby causing release of the cell sheet from the polymer. In a particular embodiment, this polymer covers the surface of a substrate such as a Petri dish and is immobilized on it. Therefore, at temperatures above 31° C., the polymer on the substrate surface also dehydrates but since the polymer chains cover the substrate surface and are immobilized on it, the substrate surface becomes hydrophobic. Conversely, at temperatures of 31° C. and below, the polymer on the substrate surface hydrates but since the polymer chains cover the substrate surface and are immobilized on it, the substrate surface becomes hydrophilic. The hydrophobic surface is an appropriate surface for the adhesion and growth of cells, whereas the hydrophilic surface inhibits the adhesion of cells and the cells are detached simply by cooling the culture solution.

In some embodiments, the substrate surface of the cell culture support is coated with a protein selected from the group consisting of: type I collagen, laminin, fibronectin, nidogen, Matrigel, and heparan sulfate proteoglycan.

The chondrocytes and chondroprogenitor cells may be added to the culture solution on the temperature-responsive polymer in the cell culture support at various cell densities to optimize formation of the cell sheet or its characteristics. For example, in some embodiments the initial cell density of the chondrocytes in the cell culture support used for preparation of the cell sheet is from 1×10³/cm² to 5×10⁶/cm². In some embodiments, the initial cell density of the chondrocytes in the cell culture support is at least 1×10³, 1×10⁴, 1×10⁵, 1.5×10⁵, 2×10⁵, 3×10⁵, 4×10⁵, 5×10⁵, 6×10⁵, 7×10⁵, 8×10⁵, 9×10⁵, 1×10⁶, 1.5×10⁶, 2×10⁶, 3×10⁶, 4×10⁶, or 5×10⁶ cells/cm². Any of these values may be used to define a range for the initial cell density of the chondrocytes in the cell culture support. For example, in some embodiments, the initial cell density in the cell culture support is from 1×10³ to 5×10⁶ cells/cm², 1×10⁴ to 5×10⁶ cells/cm², or 1×10⁵ to 5×10⁶ cells/cm².

The chondrocytes may be cultured on the culture support for at least 1, 2, 3, 4, 5, 6, 7, 8, 9, 10, 11, 12, 13, 14, 15, 16, 17, 18, 19, 20, 21, 22, 23, 24 or 25 days before adjusting the temperature of the culture solution to below the lower critical solution temperature (adjusting step b), and detaching the cell sheet from the culture support (detaching step c). In a particular embodiment, the chondrocytes are cultured for 13 to 25 days before adjusting the temperature of the culture solution and detaching the cell sheet from the culture support. In some embodiments, the adjusting step is performed when the chondrocytes are confluent.

The chondrocyte cell sheet may be prepared in a range of different sizes depending on the application. In some embodiments, the chondrocyte cell sheet has a diameter of at least 1, 2, 3, 4, 5, 6, 7, 8, 9, 10, 15 or 20 cm. Any of these values may be used to define a range for the size of the chondrocyte cell sheet. For example, in some embodiments, the chondrocyte cell sheet has a diameter from 1 to 20 cm, from 1 to 10 cm or from 2 to 10 cm. In some embodiments, the chondrocyte cell sheet has an area of at least 1, 2, 3, 4, 5, 6, 7, 8, 9, 10, 15, 20, 30, 40, 50, 60, 70, 80, 90, 100, 150, 200, 250 or 300 cm². Any of these values may be used to define a range for the size of the chondrocyte cell sheet. For example, in some embodiments, the chondrocyte cell sheet has an area from 1 to 100 cm², 3 to 70 cm², or 1 to 300 cm². The methods described herein result in a chondrocyte cell sheet in which the surface area of the cell sheet is much greater than its thickness. For example, in some embodiments the ratio of the surface area of the chondrocyte cell sheet to its thickness is at least 10:1, 100:1, 1000:1, or 10,000:1. The chondrocyte cell sheets described herein comprise one or more layers of confluent chondrocytes, for example, 1, 2, 3, 4, 5, 6, 7, 8, 9 or 10 layers of chondrocytes. In some embodiments, the chondrocyte cell sheet comprises fewer than 2, 3, 4, 5, 6, 7, 8, 9 or 10 layers of chondrocytes. In some embodiments, the chondrocyte cell sheet comprises at least 1, 2, 3, 4, 5, 6, 7, 8, 9 or 10 layers of chondrocytes.

In some aspects, the disclosure also relates to a chondrocyte cell sheet produced by any of the methods described herein.

### IV. Methods of Treatment

The chondrocyte cell sheets described herein can be transplanted to a subject by applying the cell sheet to a tissue (e.g. cartilage tissue) in the subject. For example, as disclosed in the Examples below, when a chondrocyte cell sheet was prepared by the methods described herein and implanted into a subject, hyaline cartilage tissue regeneration was observed.

Accordingly, in some aspects, the present disclosure relates to a method of transplanting a chondrocyte cell sheet to a subject comprising applying a chondrocyte cell sheet as described herein to a tissue of a subject. In a particular embodiment, the subject is a human. A support membrane may be used to transfer the chondrocyte cell sheet to the tissue of the subject. The support membrane can be, for example, poly(vinylidene difluoride) (PVDF), cellulose acetate, and cellulose esters. The chondrocyte cell sheets readily adhere to target tissue, self-stabilizing without suturing after being placed directly onto the target tissue for a short period of time. For example, in some embodiments, the chondrocyte cell sheet adheres to the target tissue within 5, 10, 15, 20, 25, or 30 minutes after contact with the tissue. Once the chondrocyte cell sheet has adhered to the target tissue, the support membrane may be excised. In some embodiment, the chondrocytes in the cell sheet are autologous to the subject, i.e. isolated from the same subject to which the cell sheet is applied. In certain embodiments, the chondrocytes in the cell sheet are allogeneic to the subject, i.e. are isolated from a different individual from the same species as the subject, such that the genes at one or more loci are not identical.

In certain aspects, the present disclosure relates to a method of generating cartilage tissue in a subject, the method comprising applying one or more cell sheets as described herein to cartilage tissue in a subject. Applying the one or more chondrocyte cell sheets to the cartilage tissue may result in regeneration of new cartilage tissue, e.g. hyaline cartilage tissue.

In certain aspects, the present disclosure relates to a method of treating a disorder in a subject, the method comprising applying one or more cell sheets as described herein to cartilage tissue in a subject. In some embodiments, the disorder is selected from the group consisting of: a cartilage partial defect, a cartilaginous injury and an osteochondral injury.

In some embodiments, the chondrocytes in the cell sheet are autologous to the subject. In some embodiments, the chondrocytes in the cell sheet are allogeneic to the subject.

In some embodiments at least 2, 3, 4, 5, 6, 7, 8, 9 or 10 chondrocyte cell sheets are applied to cartilage tissue in the subject. In a particular embodiment, two chondrocyte cell sheets are applied to the cartilage in the subject. As discussed above, the two or more cell sheets may be stacked on top of each other and cultured for one or more days to allow for further differentiation of the cell sheets before transplantation to the subject.

In particular embodiments of the aforementioned methods, the subject to which the chondrocyte cells sheet is applied is a human.

### EXAMPLES

### Example 1. In Vitro Characterization of Juvenile Cartilage-Derived Chondrocytes and Preparation of Chondrocyte Cell Sheets

### Methods

Cartilage Sampling from Juvenile Human Polydactyly Donors

Cartilage from the phalanx and metacarpal bones of amputated polydactylous fingers and toes from 12 juvenile patients (ages ranging from 7 to 48 months) was sharply dissected using a scalpel and maintained in saline immediately following harvest.

### Chondrocyte Isolation

Cartilage harvested from juvenile donor tissues was transferred into saline, cut into <1 mm² pieces by scalpel, and then incubated with 5 mg/mL of Type 1 collagenase at 37° C. for 1.5-3.0 hours (LS004197, Worthington Biochemical, Lakewood, USA). Resulting cells were filtered through a 100-μm cell strainer, washed with saline and then re-suspended in chondrocyte culture medium (DMEM-F12, 11320082, ThermoFisher Scientific, Waltham, USA) containing 1% antibiotic-antimycotic (15240062, ThermoFisher) and 20% fetal bovine serum (FBS) (16000044, ThermoFisher).

### Cell Culture

Isolated chondrocytes were seeded on polystyrene dishes (CELLTREAT, Pepperell, USA) at 5,000-10,000 cells/cm² in chondrocyte culture medium (described above). Medium was replaced with chondrocyte medium supplemented with 100 μg/mL L-ascorbic acid phosphate magnesium salt n-hydrate (013-19641, Fujifilm Wako Pure Chemical, Osaka, Japan) at the first medium change on day 4. Cells were passaged with this medium thereafter with daily observation by phase contrast microscopy throughout cell culture. Subconfluent cells were collected by TrypLE Select (12563011, ThermoFisher) dissociation and counted. Expanded cells were cryopreserved in STEM-CELLBANKER GMP grade (Zenoaq, Fukushima, Japan) at the end of P0. Serial subculture was performed with the thawed cells at the initial density of 10,000 cells/cm² passaged every 3-5 days.

### Juvenile Chondrocyte Sheet Preparation

Cell sheets were prepared from passage 1 cells sourced from thawed cryopreserved cells. Subconfluent P1 cells were collected with 1×TrypLE Select for 5 min, then seeded at a density of 10,000 cells/cm² on temperature responsive cell culture inserts (CellSeed, Tokyo, Japan). Chondrocyte culture media was changed every 3-4 days. After 2 weeks of culture, cell sheets were harvested manually with forceps after incubation at room temperature.

### Cell Viability and Total Cell Number of Chondrocyte Sheet

Single cells from fabricated JCC sheets were isolated by incubation with TrypLE Select for 15 min and 0.25 mg/mL collagenase P (11 213 857 001, Roche, Basel, Switzerland) for 30 min.

Cells were counted via hemocytometer and cell viability was demonstrated via trypan blue (T8154, MilliporeSigma) dye exclusion.

### Chondrogenic Differentiation Culture

JCCs harvested at the end of P2 and P9 cultures or isolated cells from JCC sheets were aliquoted in chondrocyte culture medium at 2.5×105 12 cells in 15 mL conical tubes for pellet cultures. Tubes were spun at 500×g for 10 minutes. Caps were loosened and cells were incubated at 370 C, 5% CO₂ for 3 days to facilitate pellet formation. After the 3-day incubation step, chondrogenic samples were induced with chondrogenic medium, control samples were replaced with new chondrocyte culture medium, and all samples were transferred to a hypoxia incubator (370 C, 5% CO₂, 5% O₂). Chondrogenic medium contained HG-DMEM supplemented with 10 ng/mL transforming growth factor beta-3 (TGFβ3) (ThermoFisher), 200 ng/mL bone morphogenic protein-6 (BMP6) (PeproTech), 1% Insulin-Transferrin-Selenium (ITS-G) (ThermoFisher), 1% PS (Life Technologies), 1% non-essential amino acids (NEAA) (ThermoFisher), 100 nM dexamethasone (MP Biomedicals, Irvine, USA), 1.25 mg/ml bovine serum albumin (BSA) (MilliporeSigma), 50 μg/mL L-ascorbic acid 2-phosphate (MilliporeSigma), 40 μg/mL L-proline (MilliporeSigma), and 5.35 μg/mL linoleic acid (MilliporeSigma). Media was changed twice a week for 3 weeks.

### In Vitro Cell Transformation Assay

In vitro cell transformation was evaluated by serial passage culture and soft agar assay. Juvenile chondrocytes were passaged up to 13 passages (57-67 days in total) with a seeding density of 10,000 cells/cm². Cells were observed every day and cell growth rate was calculated as doubling time. Cell transformation was assessed by detecting anchorage-free proliferation. Cultured cells isolated from P2 chondrocyte sheets of normal culture periods (2 weeks) and extended culture periods (3.5 weeks) were seeded in soft agar gel with chondrocyte culture media with ascorbate at a density of 5,000 cells per well by using CytoSelect Cell Transformation Assay (CBA-9 140, Cell Biolabs, San Diego, USA). Fluorescent signal representing cell number was measured with a spectrofluorometer (Cytation 3 image reader, BioTek, Winooski, USA) on day 0 and day 8 according to manufacturer's protocol. HepG2 cells (HB-8065, ATCC) in DMEM containing 10% FBS and 1% antibiotic-antimycotic were used as positive control for anchorage-free cell growth. Averages of relative fluorescent units from duplicate or triplicate assays are shown.

### Flow Cytometry

Isolated cell suspensions from chondrocyte sheets (dissociated as described before) were aliquoted and incubated in 1 μg/mL Fc block solution (564220, BD, Franklin Lakes, USA), resuspended in 10% FBS-containing PBS for 5-10 min, then labeled with fluorescent-conjugated antibodies (Supplementary Table 1) for 15 minutes with brief vortexing steps. Cells were washed with 10% FBS-containing PBS, centrifuged, resuspended with 1:1000 propidium iodide (PI) (556463, BD) in 10% FBS-containing PBS. Samples were analyzed with a cell analyzer (Canto, BD). Doublets were excluded with FSC-W and SSC-W gating, then the PI-negative population was analyzed.

### Histology

Fabricated cell sheets were fixed with 4% paraformaldehyde for 30 min. Harvested rat knee tissue was fixed in 4% paraformaldehyde for four days and decalcified in RapidCal Immuno (BBC Biochemical, Mount Vernon, USA) for one day. Samples were embedded in paraffin blocks and then cut into 5-μm transverse sections with a microtome. Slides were deparaffinized by baking in an oven at 65° C. and subsequent washes with xylene and ethanol. Sections were hydrated by gradual ethanol replacement by distilled water. Safranin-O was used for metachromatic staining for sulfated glycosaminoglycans. Samples were stained for 5 min with Wiegert's Iron Hematoxylin (MilliporeSigma), 5 min with 0.5 g/L Fast Green FCF (MilliporeSigma), and 5 min with 0.1% Safranin-O (MilliporeSigma). Images were taken with a BX41 microscope (Olympus, Tokyo, Japan) and AmScope Software (USA).

### Immunohistochemistry

Sections of histology samples were hydrated, then antigen retrieval was performed. The retrieval method was chosen to preserve the tissue integrity of knee samples and cell sheet samples after the staining optimization: protease K (S3020, Agilent Technologies, Santa Clara, USA) for COL2 and vimentin staining of knee samples; heat antigen retrieval in citrate buffer (pH 6.0) (C9999, MilliporeSigma, Burlington, USA) for COL2 staining of cell sheet samples. Peroxidase blocking was performed with Hydrogen Peroxide Blocking Reagent (ab64218, Abcam, Cambridge, UK). After blocking with 5% donkey serum and 0.1% Triton-X in PBS for one hour. Samples were then incubated overnight with primary antibodies at 4° C. Polyclonal goat anti-type I collagen (1:200, SouthernBiotech, Birmingham, USA), monoclonal mouse anti-type II collagen (1:200, 2B1.5, ThermoFisher), polyclonal goat anti-aggrecan (1:100, AF1220, R&D Systems, Minneapolis, USA), and monoclonal rabbit anti-human vimentin (1:200, SP20, Abcam) were used as primary antibodies. Normal mouse IgG2a (X0943, Agilent), normal goat IgG (NI02, MilliporeSigma), or normal rabbit IgG (X0903, Agilent) were used as isotype controls at the same concentration as the primary antibodies. Horseradish peroxidase (HRP)-conjugated goat anti-mouse antibody (1:1,000, 115-035-166, Jackson ImmunoResearch, West Grove, USA) was used for type II collagen. HRP-conjugated donkey anti-goat antibody (1:1,000, 705-035-147, Jackson) was used for type I collagen and aggrecan staining. HRP-conjugated goat anti-rabbit antibody (1:1000, 111-035-144, Jackson) was used for human vimentin staining. ImmPACT DAB Peroxidase (HRP) Substrate (SK-4105, Vector Laboratories, Burlingame, USA) was used as a chromogen. Brightfield images were taken with a BX41 microscope and AmScope Software. Fluorescent images were taken with Axio Vert.A1 microscope and ZEN software (Zeiss, 10 Oberkochen, Germany).

### Results

### In Vitro Characterization of Juvenile Cartilage-Derived Chondrocytes

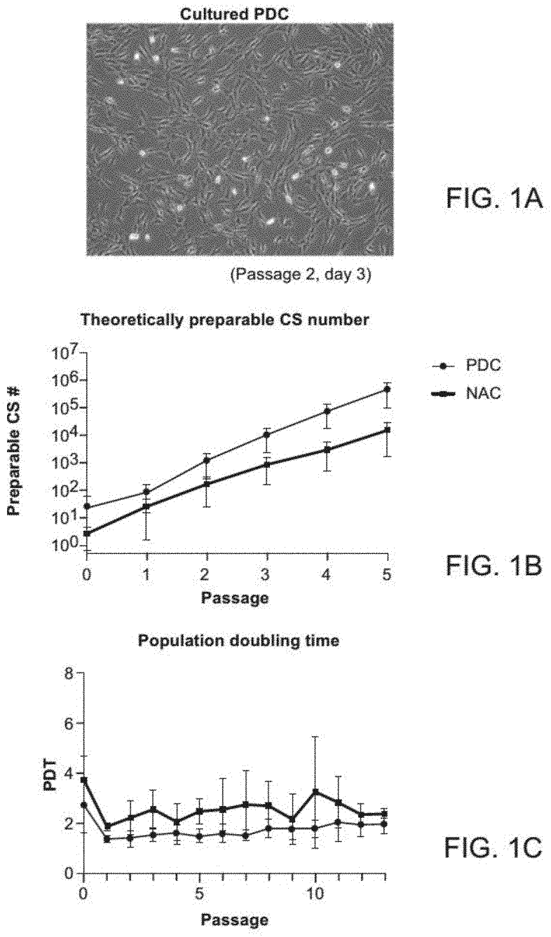

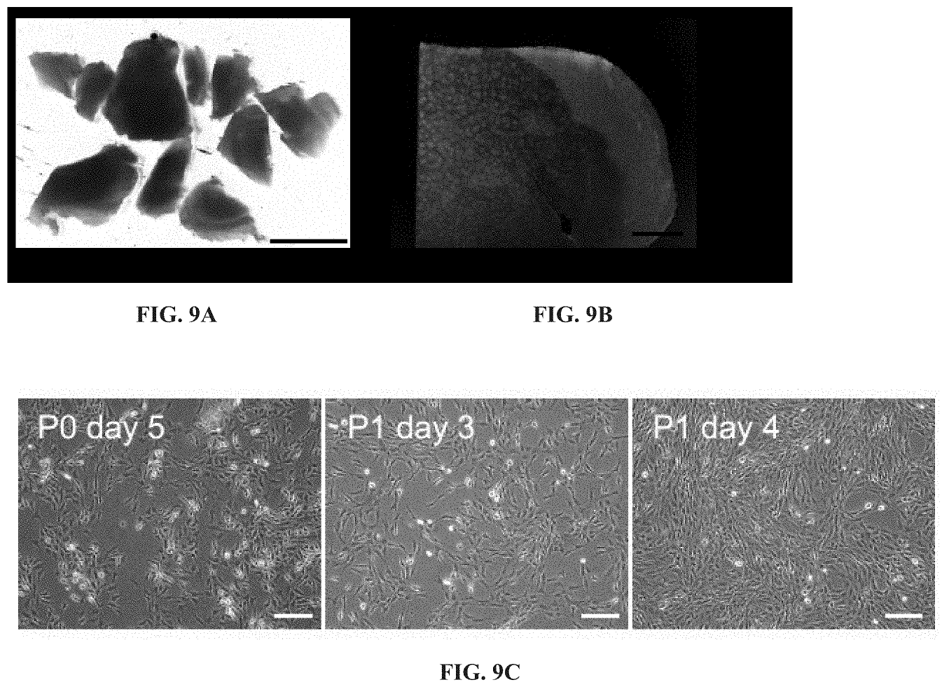

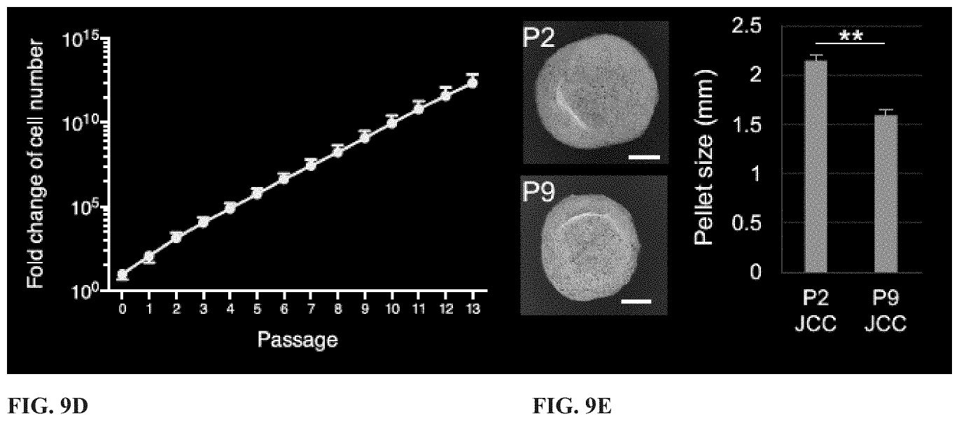

To demonstrate safety of juvenile cartilage-derived chondrocyte (JCC) sheets, juvenile cartilage tissue and JCCs in culture were characterized. Surgically discarded polydactyly cartilage samples from 12 juvenile human donors were harvested (FIG. 9A) and confirmed to contain hyaline cartilage using safranin-O staining (FIG. 9B). The morphology of isolated chondrocytes was observed after initial attachment and during subsequent culture. The chondrocytes showed stellate shapes after surface attachment, then spread after one passage (FIGS. 1A and 9C). These cells exhibit a constant growth rate for over 10 passages (FIGS. 1B and 9D). The theoretical number of PDC sheets that could be prepared from the cultured cells was calculated based on the observation that 42,000 cells (10,000 cells/cm²) are needed to prepare one cell sheet. Theoretical numbers of PDC sheets prepared from cultured cells at a given passage are shown in FIG. 1C. At all passages, PDC samples are able to produce a greater number of cell sheets compared to NAC. Harvested JCCs at the end of Passage 2 (P2) and P9 both formed fully differentiated hyaline cartilage pellets after 3-week differentiation culture although the size of P9 pellets were slightly smaller compared to P2 pellets (FIG. 9E), suggesting decreased growth potential, but maintained differentiation potential at P9.

### In Vitro Characterization of Juvenile Cartilage Derived Chondrocyte Sheets

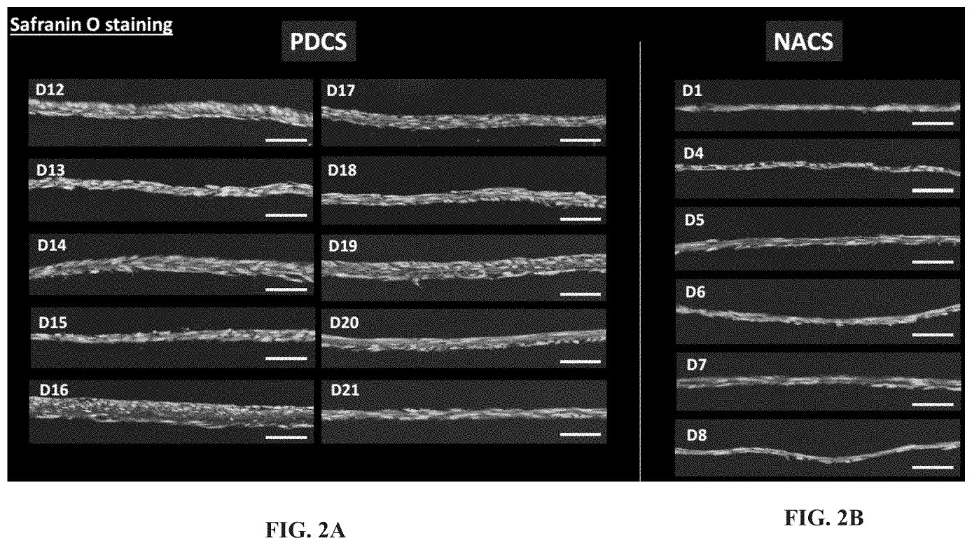

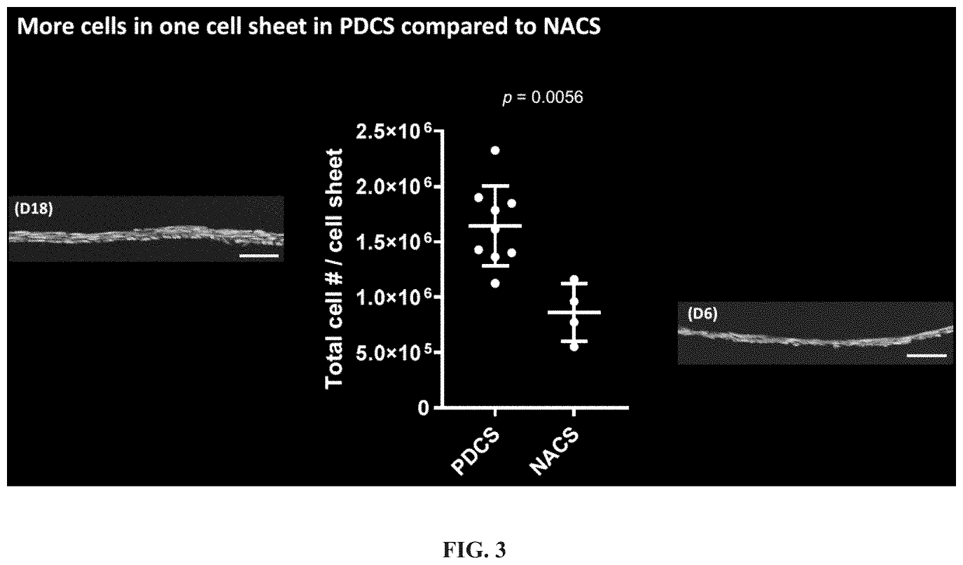

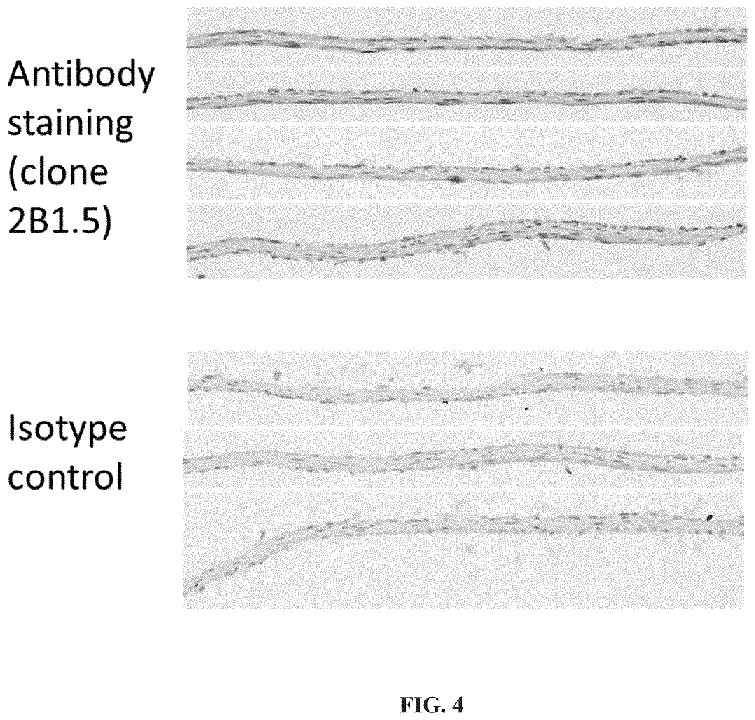

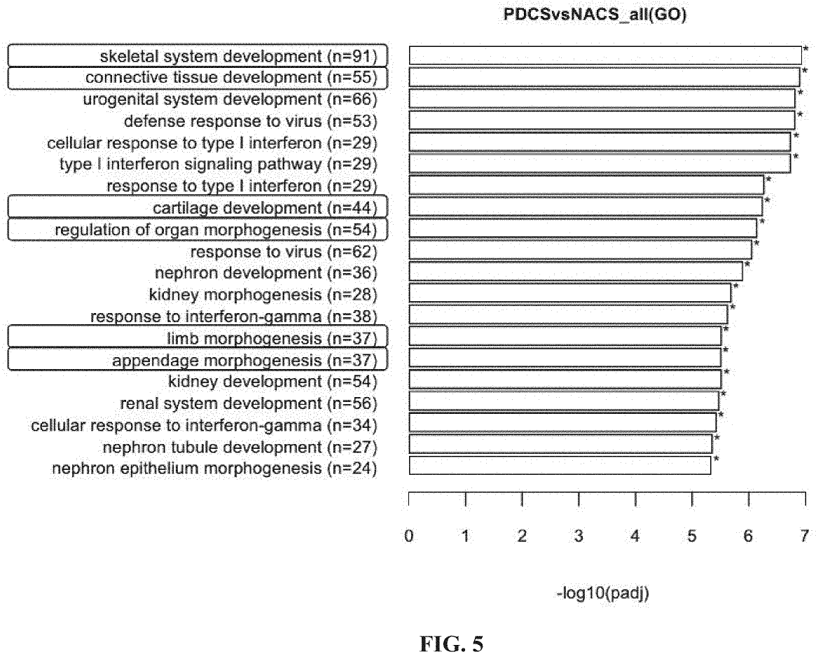

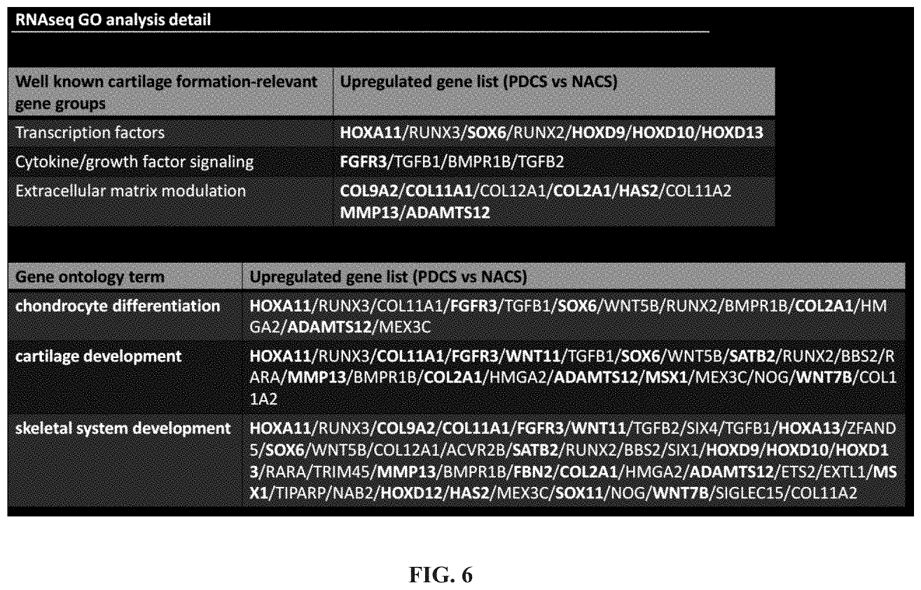

Passage 2 (P2) PDCs cultured on temperature-responsive cell culture insert for 2 weeks were confluent and able to be harvested as cell sheets. Histological samples of the PDC sheets showed multi-layer structure, including at least three cells in a vertical direction whereas NAC sheets showed single or two layers of cells in a sheet (FIG. 2B). PDC sheets contain approximately twice the cell number in each sheet compared to normal adult cartilage derived cell sheets (FIG. 3). The PDC cell sheets were positive for type II collagen (FIG. 4). A normal mouse immunoglobulin was used as a staining isotype control.

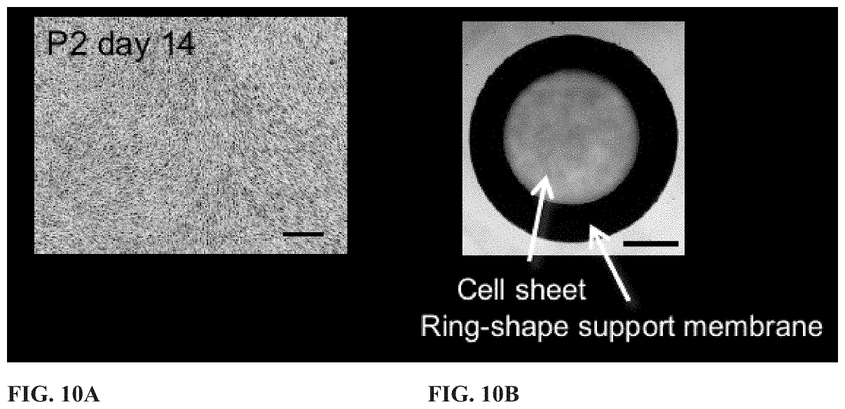

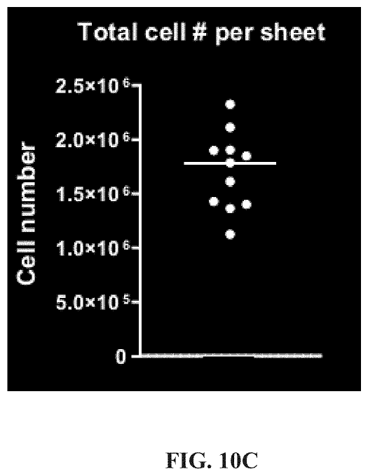

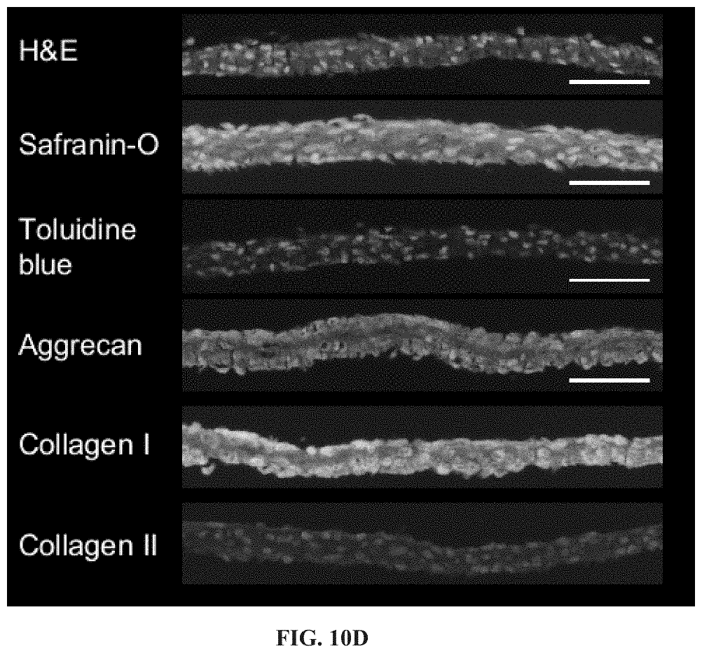

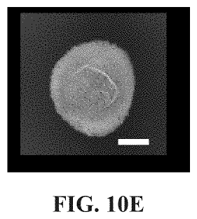

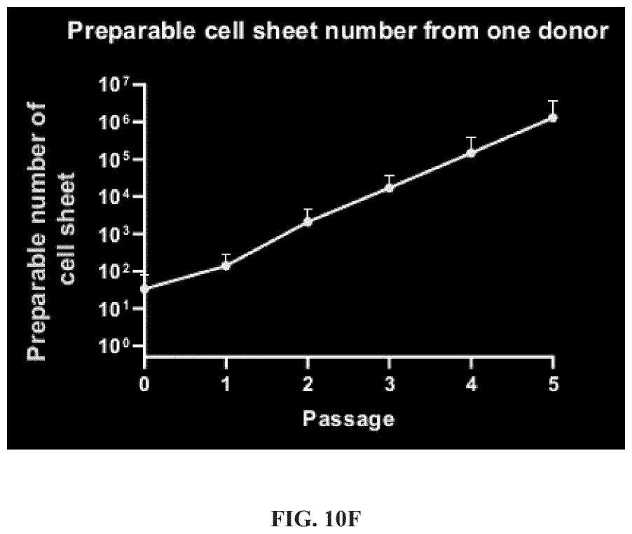

P2 JCCs cultured on temperature-responsive cell culture insert for 2 weeks were confluent (FIG. 10A) and able to be harvested as cell sheets (FIG. 10B). JCC sheets maintain high cell viability (98.0±1.3%) and rich cell numbers in each sheet construct (1.90±0.48×10⁶ cells per sheet) (FIG. 10C). After detachment, JCC sheets undergo a spontaneous, endogenous contraction resulting in a multi-cell thick sheet structure without folding (FIG. 10D). The cell sheet stains negatively for safranin-O and toluidine blue (only at nuclei), but positively for aggrecan and type I collagen with immunohistochemistry (FIG. 10D). The expression of type II collagen was not evident in the cell sheets. These in vitro characters were maintained in the cell sheets prepared at passage 9. Importantly, cells isolated from JCC sheets exhibit robust chondrogenic capacity in pellet cultures (FIG. 10E). Theoretical numbers of JCC sheets prepared from a single polydactyly donor at a given passage are shown in FIG. 10F.

### In Vitro Safety Evaluation of Juvenile Cartilage-Derived Chondrocyte Sheets

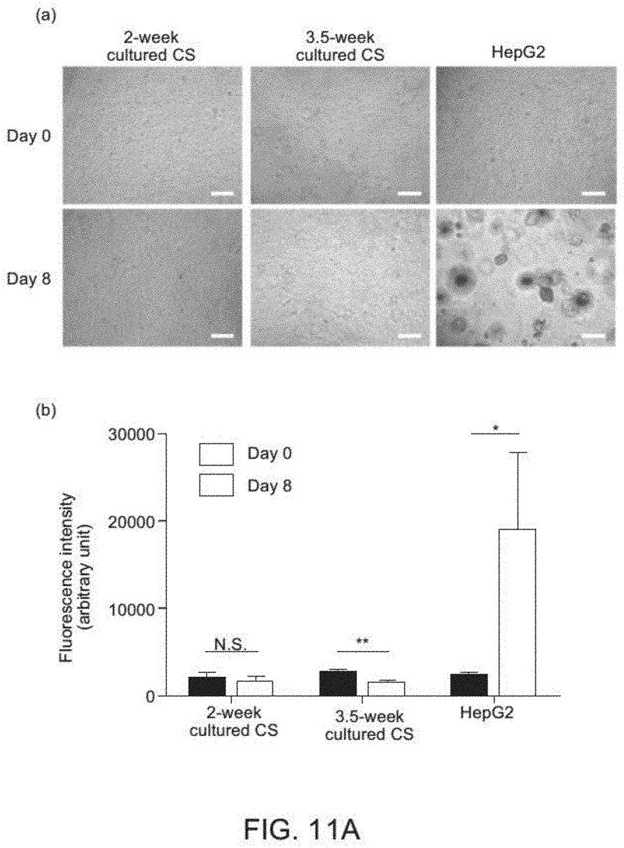

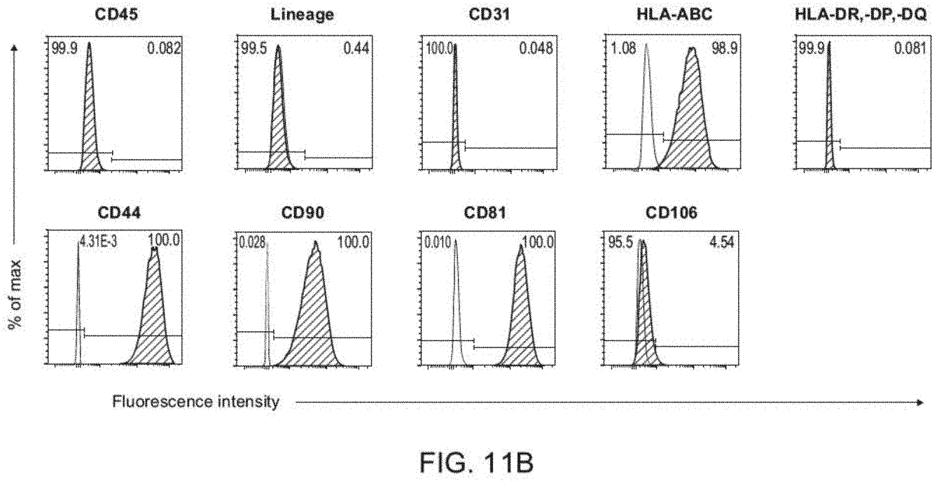

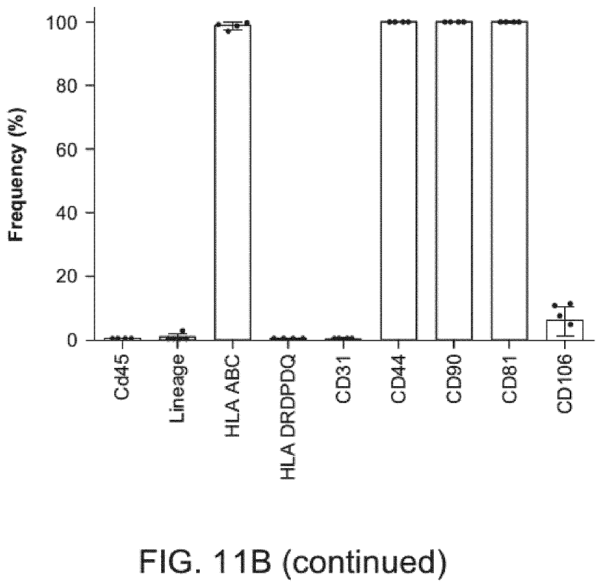

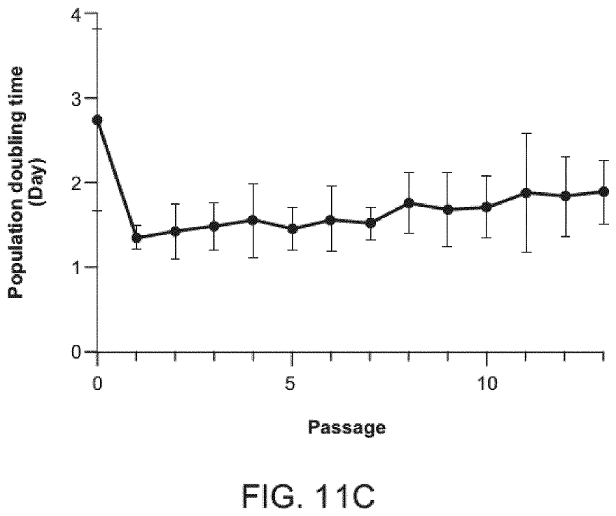

JCC isolated from sheets prepared from four individual donors showed no anchorage-independent colony growth in 10,000 seeded cells (<0.01%) (FIG. 11A(a)). Cells from both regular duration (2-week) cultured cell sheets and extended (3.5-23 week) cultured JCC sheets showed no increased signal after 8-day soft agar culture, suggesting no anchorage-independent cell growth, whereas HepG2 cells (positive control) showed significant cell growth after 8 days (FIG. 11A(b)). Cell surface markers for leukocytes (CD45 and lineage cocktail) and vascular endothelial cells (CD31) were negligible (FIG. 11B), indicating that the isolation and culture processes are free of contaminants. MHC-I molecules, HLA-ABC, were expressed 100% whereas MHC II molecules, HLA-DR, -DP, -DQ, were not detected in the cells isolated from JCC sheets (FIG. 11B), suggesting low allogeneic immunogenicity in long-term graft retention. Expression of mesenchymal cell markers, CD44, CD90 and CD81, were 100%, suggesting pure populations of cultured chondrocytes. By contrast, variability of CD106 expression among donors was observed (5.6±4.6%), indicating CD106 cannot be used as a purity marker. To establish phenotypic stability, JCC were serially passaged and morphology and population doubling times at each passage were evaluated. Doubling time of cell growth was stable in serial subculture up to PI3 (FIG. 11C), which strongly suggests that chondrocytes in sheets are highly unlikely to transform to infinitely proliferative cells in a further scaled production process.

### Transcriptome Analysis

Global gene expression in PDC and NAC cell sheets was analyzed with RNAseq. Enriched gene ontology (GO) comparing PDC sheets and NAC sheets shows multiple pro-chondrogenic GO terms (FIG. 5, gated). As shown in FIG. 5, multiple genes in several classes including genes involved in cartilage development, skeletal system development, and connective tissue development were differentially regulated in PDC cell sheets relate to NAC cell sheets (“n” indicates the number of genes in each class that were differentially regulated; the bars indicate the statistical significance that is calculated based on the ratio of the differentially expressed gene number/total gene number in each class). Examples of differentially expressed genes in each gene type and GO term are shown in FIG. 6. These differentially expressed genes are potential therapeutic surrogate markers for PDC sheets.

### Example 2. Evaluation of Safety and Efficacy of PDC Cell Sheets In Vivo Methods

### Surgical and Transplantation Procedure

The animal study plan was evaluated and approved by Institutional Animal Care & Use Committee (IACUC) (assigned ID: 17-09011). Sprague Dawley (SD) rats and nude rats at the age of 6 weeks, male and female, were purchased from Charles River Laboratories, Wilmington, MA. After a week of acclimatization at animal facility, the animals were anesthetized using isoflurane and O₂ gas. A medial parapatellar incision was made to expose the knee joint; the patella was laterally dislocated and a focal chondral defect (diameter 2 mm; depth 200-350 μm) was created on the patellar groove of the femur using an electric grinder and biopsy punch without damaging subchondral bones. Defect depth was controlled by the procedure under a surgical stereo zoom microscope (Olympus, Japan) with repeated depth measurement with a needle tip (25G, BD). Chondrocyte sheets prepared in temperature responsive cell culture substrate in 6 well plates were washed with saline, then cut into half and transplanted on the knee after the defect creation. The animal received painkillers; buprenorphine for 2 days and carprofen for 3 days in compliance with IACUC protocol. Animals were sacrificed after 4 weeks for histological evaluation.

### Weight Bearing Distribution

Weight distribution was tested with Incapacitance Tester (Linton), a device with two separate boards on which the rats sit and measures how the rats distribute their weight. All animals were acclimatized 1-2 times before and after surgery. No measurement was done until 2-week time point after surgery to avoid the effect of muscle trauma and painkiller. The weight distribution was calculated by the following formula.

Weight distribution (%)=(Treated side's load)/(Treated side's load+intact side's load)*100

Sample numbers at week 2, 3, 4, 5, and 6=13, 12, 12, 8, and 8 (defect only group); =16, 15, 15, 9, and 9 (defect+CS group), respectively. Data was shown as mean and SEM. **p<0.01, *p<0.05 by Welch's t-test.

### Results

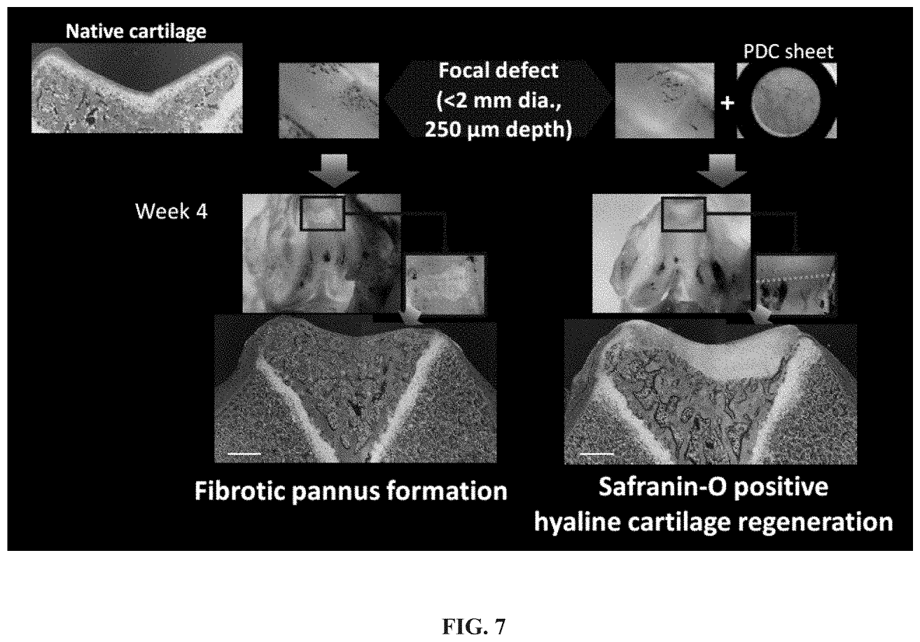

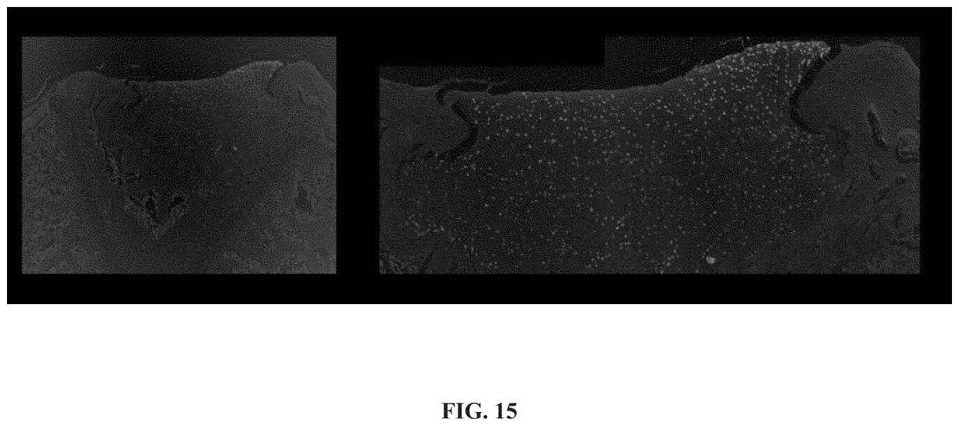

To demonstrate both safety and efficacy in vivo, PDC sheets were transplanted at the time of surgical focal osteochondral defect creation in rats. The two experimental groups, (PDC sheet treatment group and defect-only negative control group) were observed under stereomicroscopy, and histologically examined and compared post-transplantation. An immunocompromised athymic rat model was selected to evaluate sheet-induced neocartilage formation. Seven-week-old nude rats received focal osteochondral defects in the trochlear groove (2-mm diameter, 200-350 μm depth. Depressed knee surface and fibrotic pannus indicative of failure to spontaneously regenerate cartilage tissue was observed in the defect only group at all time points (4, 8, 12, 24 weeks) (FIG. 7). Interestingly, in contrast to the defect only group, complete fill regenerated white cartilage occurred in defect areas of the PDC sheet treatment group at all time points (FIG. 7). Histological analysis showed the samples of defect only group are safranin-O negative, whereas the PDC sheet treatment group exhibited safranin-O positive, thick hyaline neocartilage at all time points (4, 8, 12, and 24 weeks) with a stably integrated interface with host tissue confirmed with histological analysis (FIG. 7). In addition, formation of lacuna structure was observed at all time points, suggesting that mature cartilage was formed in defect areas by 4 weeks post-transplantation, and native tissue architecture was maintained. Moreover, while regenerated cartilage was substantially thicker than rat native cartilage, no tumorigenic tissue formation was observed in all rats, suggesting safety of transplanted PDC sheets throughout 24 weeks of study. Origin of the regenerated tissue was determined using human-specific vimentin staining (FIG. 15) to distinguish human cells from rat cells.

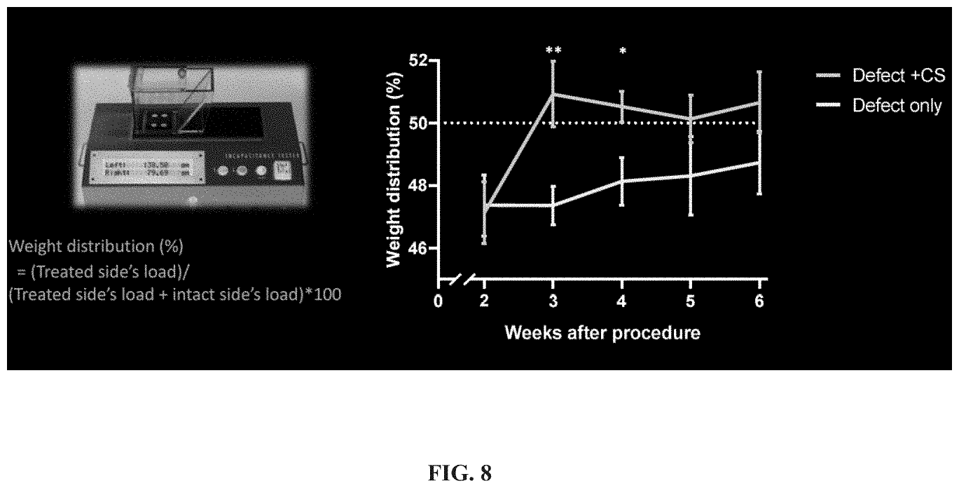

We also assessed functional recovery induced by PDC sheet transplantation on the rat focal osteochondral defect models by measuring rat hind limb weight bearing. The cell sheet transplanted group showed rapid recovery to reach equal weight distribution on each leg after 3 weeks, while the defect only group sustained low weight distribution on the injured leg for over 6 weeks (FIG. 8), indicating that regenerated cartilage tissue alleviates pain caused by the focal defect.

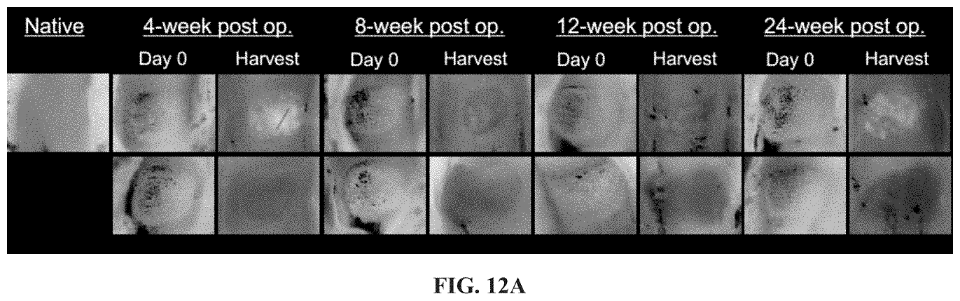

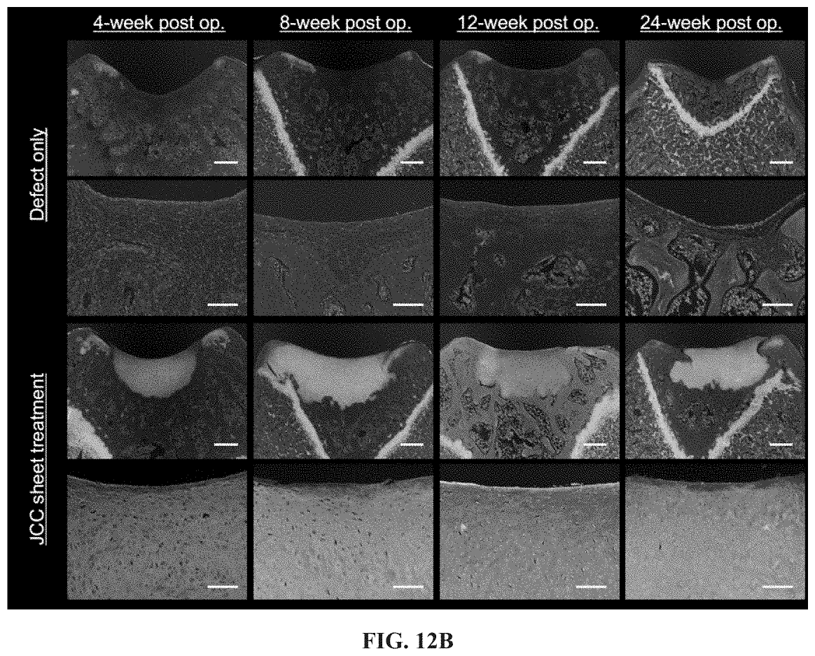

The two experimental groups, (JCC sheet treatment group and defect-only negative control group) were observed under stereomicroscopy, and histologically examined and compared post-transplantation. Preliminary JCC sheet transplantation studies using immunocompetent Sprague Dawley (SD) rats failed to regenerate cartilage. Therefore, an immunocompromised athymic rat model was selected to evaluate sheet-induced neocartilage formation. Seven-week-old nude rats received focal osteochondral defects in the trochlear groove (2-mm diameter, 200-350 μm depth. Depressed knee surface and fibrotic pannus indicative of failure to spontaneously regenerate cartilage tissue was observed in the defect only group at all time points (4, 8, 12, 24 weeks) (FIG. 12B). This is consistent with previously reported critical size defects (>1.4 mm diameter) in rat knee cartilage. Interestingly, in contrast to the defect only group, complete fill regenerated white cartilage occurred in defect areas of the JCC sheet treatment group at all time points (FIG. 12A). Histological analysis showed the samples of defect only group are safranin-O negative, whereas the JCC sheet treatment group exhibited safranin-O positive, thick hyaline neocartilage at all time points (4, 8, 12, and 24 weeks) with a stably integrated interface with host tissue confirmed with histological analysis (FIG. 12B). In addition, formation of lacuna structure was observed at all time points, suggesting that mature cartilage was formed in defect areas by 4 weeks post-transplantation, and native tissue architecture was maintained. Interestingly, the size of lacuna was smaller than host native cartilage, indicating that tissue is not the residue of original host cartilage. Moreover, while regenerated cartilage was substantially thicker than rat native cartilage, no tumorigenic tissue formation was observed in all rats, suggesting safety of transplanted JCC sheets throughout 24 weeks of study.

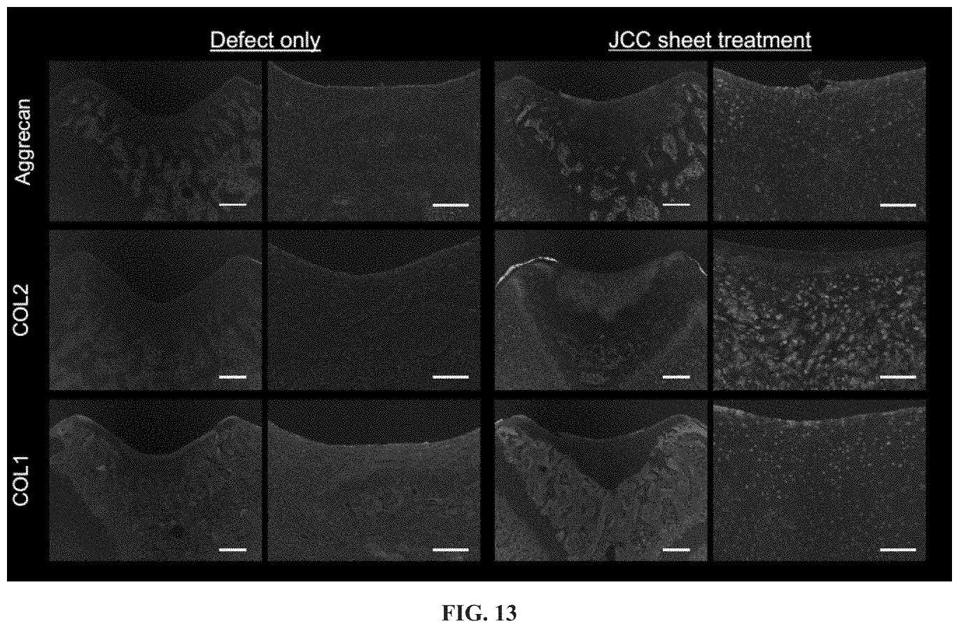

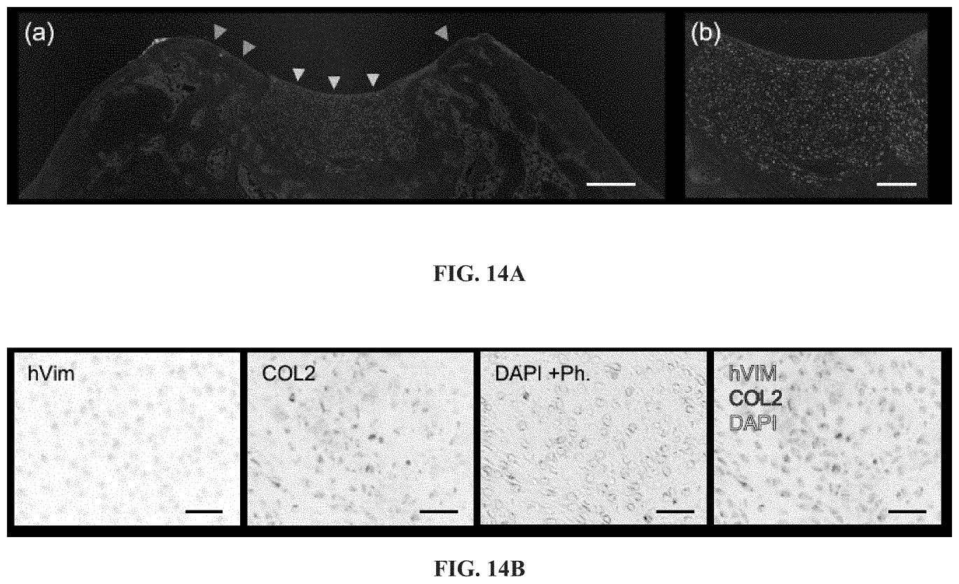

The presence of aggrecan (ACAN) and type 2 collagen (COL2), hyaline cartilage-specific matrix proteins, and type 1 collagen (COL1), a damaged or arthritic cartilage marker, was assessed on harvested knee samples by immunohistochemistry (IHC). Pannus tissue in the defect only group showed neither ACAN nor COL2 expression, but strong COL1 expression (FIG. 13A). Regenerated neocartilage in the JCC sheet treatment group demonstrated expression of ACAN and COL2 with limited expression of COL1 localized to neocartilage surfaces (FIG. 13B). Origin of the regenerated tissue was determined using human-specific vimentin staining. Specificity of human-specific anti-vimentin was confirmed on superficial cartilage of fibrotic tissue of the non-treatment group by comparison to a “pan” vimentin antibody, cross-reacting to both human and rat cartilage. Human-vimentin specific antibody did not react with the defect only samples (FIG. 14A), whereas it reacted with the neocartilage tissue areas on JCC sheet treatment samples (FIG. 14B). Interestingly, COL2 was observed at the periphery of human-vimentin positive cells and adjacent interstitial matrix (FIG. 14B). These data strongly suggest that transplanted human JCC sheets are responsible for deposition of new cartilage matrix proteins and neocartilage generation.

We assessed functional recovery induced by JCC sheet transplantation on the rat focal osteochondral defect models by measuring rat hind limb weight bearing. The cell sheet transplanted group showed rapid recovery to reach equal weight distribution on each leg after 3 weeks, while the defect only group sustained low weight distribution on the injured leg for over 6 weeks (FIG. 8), indicating that regenerated cartilage tissue alleviates pain caused by the focal defect. JCC sheet transplantation did not affect total body weight.

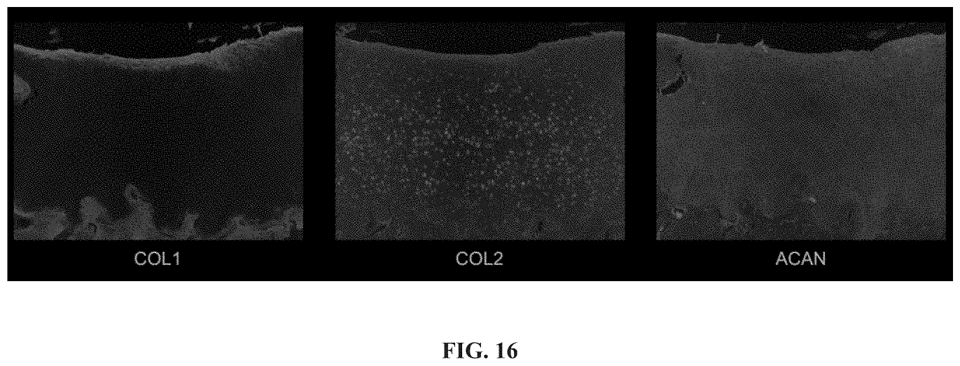

The chondrocyte cell sheets were retained on rat knees 24 weeks after transplantation and maintained expression of human vimentin (FIG. 15) and type I collagen (COL1), type II collagen (COL2) and aggrecan (ACAN) (FIG. 16).

### CONCLUSIONS

Knee cartilage does not regenerate spontaneously after injury, and a gold standard regenerative treatment algorithm has not been established. This study demonstrates preclinical safety and efficacy of scaffold-free, human juvenile cartilage-derived chondrocyte (PDC) sheets produced from routine surgical discards using thermo-responsive cultureware. PDCs exhibit stable and high growth potential in vitro over passage 10, supporting possibilities for scale-up to mass production for commercialization. PDC sheets contain highly viable, densely packed cells, show no anchorage-independent cell growth, express mesenchymal surface markers, and lack MHC II expression. In nude rat focal osteochondral defect models, stable neocartilage formation was observed at 4 weeks by PDC sheet transplantation without abnormal tissue growth over 24 weeks in contrast to the non-treatment group showing no spontaneous cartilage repair. Regenerated cartilage was safranin-O positive, contained type II collagen, aggrecan and human vimentin, and lacked type I collagen, indicating that the hyaline-like neocartilage formed originates from transplanted PDC sheets rather than host-derived cells. This study demonstrates the safety of PDC sheets and stable hyaline cartilage formation with engineered PDC sheets utilizing a sustainable tissue supply.

We claim:

1. A cell sheet for cartilage tissue repair, wherein the cell sheet is formed from cultured cells derived from cartilage tissue, wherein the cultured cells comprise chondrocytes and cells expressing transcription factors that promote differentiation into chondrocytes.

2-10. (canceled)

11. A method for producing a cell sheet for cartilage tissue repair, comprising:
(1) collecting cartilage tissue by scalpel in a clean environment, wherein the tissue includes cells containing transcription factors to differentiate into chondrocyte, from the part of cartilage tissue that appears black on an X-ray image,
(2) cutting the collected cartilage tissue with a scalpel into small pieces,
(3) collecting the cells through enzymatic treatment of the small pieces of cartilage tissue,
(4) primarily culturing the collected cells on the particular surface of material,
(5) thereafter, culturing the primarily cultured cells on a temperature responsive cell culture material, wherein a surface of the cell culture material is coated with a temperature responsive polymer which changes its hydration force within a temperature range between 0° C. and 80° C., in a temperature region wherein the polymer shows weak hydration force.
(6) detaching the cultured cells as the form of a cell sheet by adjusting the temperature of the surface of the temperature responsive cell culture material by lowering the temperature of its culture medium to a temperature at which the polymer shows a stronger hydration force.

12-22. (canceled)

23. A chondrocyte cell sheet comprising one or more layers of confluent cells comprising chondrocytes and chondroprogenitor cells, wherein the cell sheet is prepared from a mixture of cells obtained from cartilage tissue of a subject, wherein the mixture of cells obtained from the cartilage tissue comprises chondrocytes and chondroprogenitor cells.

24. The chondrocyte cell sheet of claim 23, wherein the chondrocyte cell sheet exhibits increased expression of one or more genes selected from the group consisting of ACVR2B, ADAMTS12, BBS2, BMPR1B, COL2A1, COL9A2, COL11A1, COL11A2, COL12A1, ETS2, EXTL1, FBN2, FGFR3, HAS2, HMGA2, HOXA11, HOXA13, HOXD9, HOXD10, HOXD12, HOXD13, MEX3C, MMP13, MSX1, NAB2, NOG, RARA, RUNX2, RUNX3, SATB2, SIGLEC15, SIX1, SIX4, SOX6, SOX11, TGF-β1, TGF-β2, TIPARP, TRIM45, WNT5B, WNT7B, WNT11 and ZFAND5 relative to a cell sheet prepared from cells isolated from non-polydactyl cartilage from an adult.

25. The chondrocyte cell sheet of claim 23, wherein the subject is human.

26. (canceled)

27. The chondrocyte cell sheet of claim 25, wherein the subject is 1.5 to 6 years old.

28. (canceled)

29. The chondrocyte cell sheet of claim 23, wherein the cartilage tissue of the subject is polydactyl cartilage tissue.

30-34. (canceled)

35. The chondrocyte cell sheet of claim 23, wherein the cell sheet consists essentially of chondrocytes and chondroprogenitor cells.

36. The chondrocyte cell sheet of claim 23, wherein the cell sheet comprises two or more layers of confluent cells comprising chondrocytes and chondroprogenitor cells.

37-38. (canceled)

39. A composition comprising the chondrocyte cell sheet of claim 23 and a polymer-coated culture support that is removable from the cell sheet.

40. A composition comprising at least two of the chondrocyte cell sheets of claim 23.

41. The composition of claim 40, wherein the at least two chondrocyte cell sheets are stacked on top of each other.

42. A method for producing a chondrocyte cell sheet comprising one or more layers of confluent cells comprising chondrocytes and chondroprogenitor cells, the method comprising:
a) culturing a mixture of chondrocytes and chondroprogenitor cells in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the cell culture support, thereby producing a chondrocyte cell sheet comprising two or more layers of confluent cells comprising chondrocytes and chondroprogenitor cells.

43. The method of claim 42, further comprising:
d) collecting cartilage tissue by scalpel under sterile conditions, wherein the cartilage tissue comprises chondrocytes and chondroprogenitor cells;
e) cutting the collected cartilage tissue with scalpels into pieces; and
f) collecting cells from the cartilage tissue through enzymatic treatment of the pieces of cartilage tissue.

44. The method of claim 42, wherein the adjusting step (b) is performed when the chondrocytes and chondroprogenitor cells are confluent.

45. The method of claim 42, wherein the culturing step (a) comprises adding the chondrocytes and chondroprogenitor cells to the culture solution at an initial cell density of at least 1×10⁵ cells/cm².

46. The method of claim 42, wherein the chondrocytes and chondroprogenitor cells are cultured in the culture solution on the temperature-responsive polymer for at least 2 days before the adjusting step (b).

47. The method of claim 42, wherein the cartilage tissue is derived from polydactyl cartilage tissue.

48-57. (canceled)

58. The method of claim 42, further comprising stacking at least two cell sheets on top of each other.

59. A cell sheet produced by the method of claim 42.

60. A method of generating cartilage tissue in a subject, the method comprising applying the chondrocyte cell sheet of claim 23 to cartilage tissue in a subject.

61. A method of treating a disorder in a subject, the method comprising applying the chondrocyte cell sheet of claim 23 to cartilage tissue in a subject.

62. The method of claim 61, wherein the disorder is selected from the group consisting of: a cartilage partial defect, a cartilaginous injury and an osteochondral injury.

63. (canceled)
